(19) United States
(12) Patent Application Publication
(10) Pub. No.: US 2025/0230124 A1
(43) Pub. Date: Jul. 17, 2025

(54) SMALL MOLECULE INHIBITORS OF ID PROTEINS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Robert Benezra, New York, NY (US); Ouathek Ouerfelli, New York, NY (US); Guangli Yang, New York, NY (US)

(21) Appl. No.: 18/854,039

(22) PCT Filed: Apr. 6, 2023
(86) PCT No.: PCT/US23/17695

§ 371 Date: Oct. 3, 2024

Related U.S. Application Data
(60) Provisional application No. 63/328,515, filed on Apr. 7, 2022.

(51) Int. Cl. C07C 217/62; A61K 31/137; A61K 31/343; A61K 31/444; A61P 35/00; A61P 35/04; C07C 211/52; C07D 213/38; C07D 317/58

(52) U.S. Cl. CPC C07C 217/62; A61K 31/137; A61K 31/343; A61K 31/444; A61P 35/00; A61P 35/04; C07C 211/52; C07D 213/38; C07D 317/58

(57) ABSTRACT

The present technology relates generally to compounds, compositions, and methods useful for treating, preventing, and/or ameliorating pathogenic cellular proliferation, angiogenesis, cancer, metastatic disease, and/or a pathogenic vascular proliferative disease in a subject.

### CROSS-REFERENCE TO RELATED APPLICATIONS

This application claims the benefit of and priority to U.S. Provisional Application No. 63/328,515, filed Apr. 7, 2022, the contents of which are incorporated herein by reference in their entirety for any and all purposes.

### U.S. GOVERNMENT SUPPORT

This invention was made with government support under CA008748 awarded by the National Institutes of Health. The government has certain rights in the invention.

### TECHNICAL FIELD

The present technology relates generally to compounds that inhibit “Id” (inhibitor of differentiation) proteins, compositions thereof, and methods thereof that are useful for treating, preventing, and/or ameliorating pathogenic cellular proliferation, angiogenesis, cancer, metastatic disease, and/or a pathogenic vascular proliferative disease in a subject.

### SUMMARY

In an aspect, the present disclosure provides a compound according to Formula I

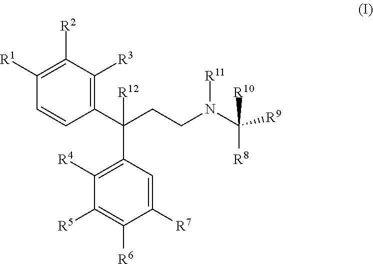

or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹³)(R¹⁴);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹⁵)(R¹⁶);
R⁸ is aryl or heteroaryl;
R⁹ and R¹⁰ are each independently H, C₁-C₃ alkyl, or trifluoromethyl;
R¹¹ is H, C₁-C₃ alkyl, aralkyl, or heteroaralkyl;
at least one of R⁹, R¹⁰, and R¹¹ is not H;
R¹² is H, C₁-C₃ alkyl, or fluoro; and
R¹³, R¹⁴, R¹⁵, and R¹⁶ are each independently C₁-C₃ alkyl.

In any embodiment herein, the compound may be

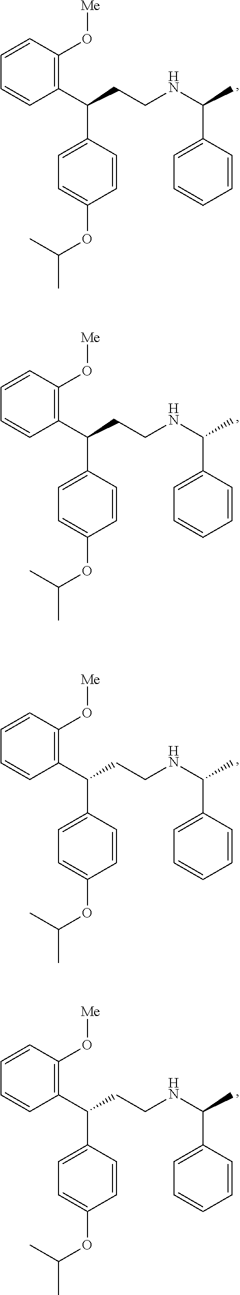

or a pharmaceutically acceptable salt and/or solvate thereof.

In a related aspect, a pharmaceutical composition is provided that includes an effective amount of a compound of Formula I for treating pathogenic cellular proliferation, angiogenesis, cancer, metastatic disease, and/or a pathogenic vascular proliferative disease in a subject; and a pharmaceutically acceptable carrier.

In an aspect, a method for treating a condition in a subject is provided, where the method includes administering a compound of Formula I to the subject in an amount effective to treat the condition, where the condition includes one or more of pathogenic cellular proliferation, angiogenesis, cancer, metastatic disease, and/or a pathogenic vascular proliferative disease.

Further aspects and embodiments of the present technology are described herein.

### BRIEF DESCRIPTION OF THE DRAWING

FIG. 1 provides the tumor volume results of a murine model of cholangiocarcinoma showing the in vivo efficacy of a compound of the present technology (AGX-502) along with a comparative compound (AGX-A), according to the working examples.

### DETAILED DESCRIPTION

It is to be appreciated that certain aspects, modes, embodiments, variations and features of the present methods are described below in various levels of detail in order to provide a substantial understanding of the present technology.

### Definitions

Unless defined otherwise, all technical and scientific terms used herein generally have the same meaning as commonly understood by one of ordinary skill in the art to which this technology belongs.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

As used herein, the terms “cancer,” “neoplasm,” and “tumor,” are used interchangeably and refer to cells that have undergone a malignant transformation that makes them pathological to the host organism. Primary cancer cells (that is, cells obtained from near the site of malignant transformation) may be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “metastasis” or “metastatic” refers to the ability of a cancer cell to invade surrounding tissues, to enter the circulatory system and to establish malignant growths at new sites.

“Non-metastatic” refers to tumors that do not spread beyond their original site of development and specifically do not enter the circulatory system and establish malignant growths at new sites.

As used herein, “prevention,” “prevent,” or “preventing” of a disease or condition refers to one or more compounds that, in a statistical sample, reduces the occurrence of the disease or condition in the treated sample relative to an untreated control sample, or delays the onset of one or more symptoms of the disease or condition relative to the untreated control sample. As used herein, prevention includes preventing or delaying the initiation of symptoms of the disease or condition. As used herein, prevention also includes preventing a recurrence of one or more signs or symptoms of a disease or condition.

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

Generally, reference to a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)CH₂, —C(CH₃)CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl(pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷³R⁷⁶ groups, wherein
R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O—. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

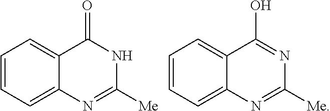

As another example, guanidines may exhibit the following isomeric forms in protic organic solution, also referred to as tautomers of each other:

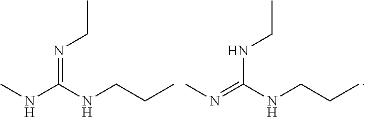

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents, and published patent specifications are referenced by an identifying citation. Also within this disclosure are Arabic numerals referring to referenced citations, the full bibliographic details of which are provided immediately preceding the claims. The disclosures of these publications, patents, and published patent specifications are hereby incorporated by reference into the present disclosure to more fully describe the state of the art to which this invention pertains.

### The Present Technology

As described in the art (for example, in U.S. Pat. Publ. No. 2009/0226422, Int'l Publ. No. WO 2015/089495, and Int'l Publ. No. WO 2021/067393, each of which is incorporated herein by reference), Id proteins have been shown to play key roles as regulators of stem cell identity in both colorectal cancer and malignant glioma, essential for both self-renewal and tumor-initiating capacity of cancer stem cells. Despite the mechanistic complexity and indefinite pathways involved in cancer stem cell development, anti-Id compounds disable Id proteins at a critical foundation to disrupt stem cell identity and impair stem cell tumor initiation. Anti-metastatic and anti-angiogenic effective anti-Id compounds may specifically target tumor stem cell viability, proliferation capacity, tumor-initiation potential, and/or cell fate determination—with the result of substantially decreasing populations of new tumor induction-competent stem cells present in new or established tumors.

The compounds of the present technology target Id1 and Id3 positive “resting” stem cells. These cells representing a pool of cancer progenitor cells relatively resistant to chemotherapy (based on their resting, non-proliferative state, such cells elude first line chemotherapy targeting proliferative cells). As such these stem cells frequently escape first line cancer treatment, whereafter they are capable of rebounding to give rise to new cancer cell populations. Yet additional evidence presented here shows that compounds of the present technology also penetrate in their effects to preclude acquired resistance, either alone or in combination with conventional chemotherapeutic cancer therapies. For example, resting stem cells or cancer cells that mutationally escape first line treatment (e.g., cells that acquire resistance to chemotherapeutic drugs through mutation) cannot further elude or develop resistance to the compounds of the present technology. Without being bound by theory, the functionally critical, evolutionarily constrained/conserved Id binding interface (targeted by the compounds of the present technology) is practically immutable to generate “acquired resistance” because any structural mutation yields a biologically inoperative Id protein, functionally null for the essential purposes such proteins serve in cancer cells.

Thus, in an aspect, the present disclosure provides a compound according to Formula I

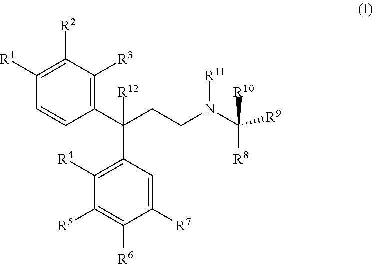

or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹³)(R¹⁴);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹⁵)(R¹⁶);
R⁸ is aryl or heteroaryl;
R⁹ and R¹⁰ are each independently H, C₁-C₃ alkyl, or trifluoromethyl;
R¹¹ is H, C₁-C₃ alkyl, aralkyl, or heteroaralkyl;
at least one of R⁹, R¹⁰, and R¹¹ is not H;
R¹² is H, C₁-C₃ alkyl, or fluoro; and
R¹³, R¹⁴, R¹⁵, and R¹⁶ are each independently C₁-C₃ alkyl.

In any embodiment herein, it may be that R¹, R², and R³ are each independently H, C₁-C₃ alkyl, C₁-C₃ alkoxy, trifluoromethyl, trifluoromethoxy, halo, or —N(R¹⁰)(R¹¹). In any embodiment herein, it may be that R¹, R², and R³ are each independently H, C₁-C₃ alkyl, C₁-C₃ alkoxy, halo, or —N(Me)₂. In any embodiment herein, it may be that R¹, R², and R³ are each independently H, methyl, methoxy, isopropyl, isopropoxy, fluoro, or —N(Me)₂. In any embodiment herein, it may be that R³ is methoxy.

In any embodiment herein, it may be that R⁴, R⁵, R⁶, and R⁷ are each independently H, C₁-C₃ alkyl, C₁-C₃ alkoxy, trifluoromethyl, trifluoromethoxy, halo, or —N(R¹²)(R¹³). In any embodiment herein, it may be that R⁴, R⁵, R⁶, and R⁷ are each independently H, C₁-C₃ alkyl, C₁-C₃ alkoxy, halo, or —N(Me)₂. In any embodiment herein, it may be that R⁴, R⁵, R⁶, and R⁷ are each independently H, methyl, methoxy, isopropyl, isopropoxy, fluoro, or —N(Me)₂. In any embodiment herein, it may be that R⁶ is isopropoxy.

In any embodiment herein, it may be that the compound is of Formula IA

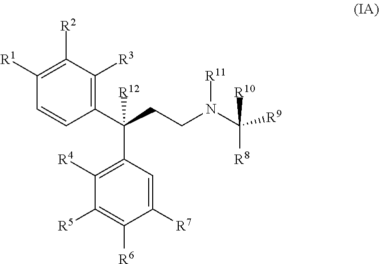

or a pharmaceutically acceptable salt and/or solvate thereof.

In any embodiment herein, it may be that the compound is of Formula IB

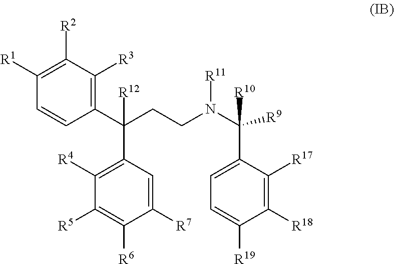

or a pharmaceutically acceptable salt and/or solvate thereof, wherein R¹⁷, R¹⁸, and R¹⁹ are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido.

In any embodiment herein, it may be that R¹⁷, R¹⁸, and R¹⁹ are each independently H, C₁-C₃ alkyl, C₁-C₃ alkoxy, trifluoromethyl, trifluoromethoxy, halo, aryloxy, aryloyl, or —N(C₁-C₃ alkyl)₂. In any embodiment herein, it may be that R¹⁷, R¹⁸, and R¹⁹ are each independently H, C₁-C₃ alkyl, C₁-C₃ alkoxy, trifluoromethyl, trifluoromethoxy, halo, or —N(Me)₂. In any embodiment herein, it may be that R¹⁷, R¹⁸, and R¹⁹ are each independently H, methyl, methoxy, isopropyl, isopropoxy, fluoro, or —N(Me)₂.

In any embodiment herein, it may be that R¹⁷, R¹⁸, and R¹⁹ are each independently H; alternatively, in any embodiment herein, it may be that at least one of R¹⁷, R¹⁸, and R¹⁹ is not H. In any embodiment herein, it may be that at least one of R¹⁷, R¹⁸, and R¹⁹ is C₁-C₃ alkoxy, trifluoromethyl, trifluoromethoxy, pentafluorosulfanyl, halo, aryloxy, aryloyl, hydroxyl, amino, or amido.

In any embodiment herein, it may be that R¹¹ is

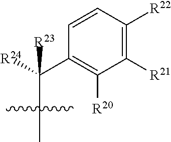

where R²⁰, R²¹, and R²² are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido; and R²³ and R²⁴ are each independently H, C₁-C₃ alkyl, trifluoromethyl, or aryl, provided at least one of R²³ and R²⁴ is not aryl.

In any embodiment herein, it may be that R²⁰, R²¹, and R²² are each independently H, C₁-C₃ alkyl, C₁-C₃ alkoxy, trifluoromethyl, trifluoromethoxy, halo, aryloxy, aryloyl, or —N(C₁-C₃ alkyl)₂. In any embodiment herein, it may be that R²⁰, R²¹, and R²² are each independently H, C₁-C₃ alkyl, C₁-C₃ alkoxy, trifluoromethyl, trifluoromethoxy, halo, or —N(Me)₂. In any embodiment herein, it may be that R²⁰, R²¹, and R²² are each independently H, methyl, methoxy, isopropyl, isopropoxy, fluoro, or —N(Me)₂.

In any embodiment herein, it may be that R²⁰, R²¹, and R²² are each independently H; alternatively, in any embodiment herein, it may be that at least one of R²⁰, R²¹, and R²² is not H. In any embodiment herein, it may be that at least one of R²⁰, R²¹, and R²² is C₁-C₃ alkoxy, trifluoromethyl, trifluoromethoxy, pentafluorosulfanyl, halo, aryloxy, aryloyl, hydroxyl, amino, or amido.

In any embodiment herein, it may be that the compound is

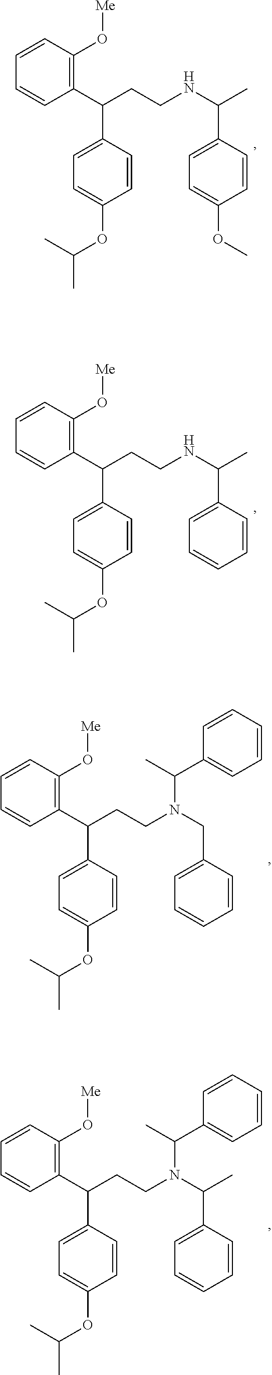 [SMILES: COc1ccc(C(C)NCCC(c2ccc(OC(C)C)cc2)c2ccccc2OC)cc1 ; COc1ccccc1C(CCNC(C)c1ccccc1)c1ccc(OC(C)C)cc1 ; COc1ccccc1C(CCN(Cc1ccccc1)C(C)c1ccccc1)c1ccc(OC(C)C)cc1 ; COc1ccccc1C(CCN(C(C)c1ccccc1)C(C)c1ccccc1)c1ccc(OC(C)C)cc1]

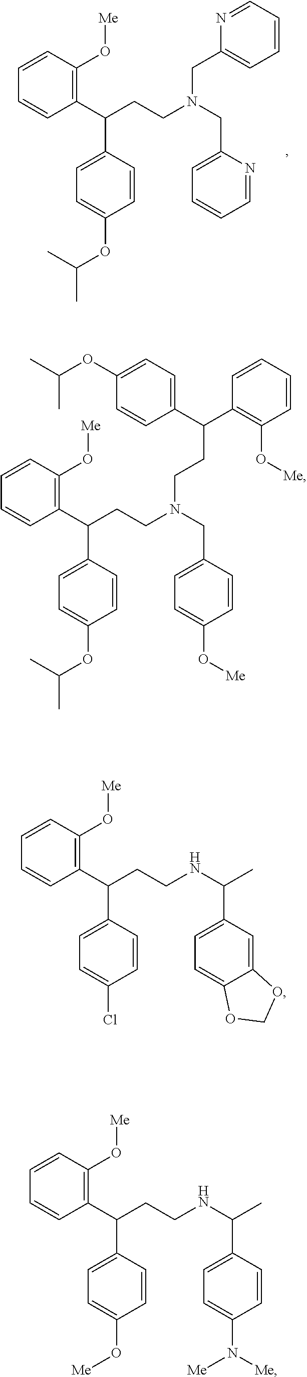 [SMILES: COc1ccccc1C(CCN(Cc1ccccn1)Cc1ccccn1)c1ccc(OC(C)C)cc1 ; COc1ccc(CN(CCC(c2ccc(OC(C)C)cc2)c2ccccc2OC)CCC(c2ccc(OC(C)C)cc2)c2ccccc2OC)cc1 ; COc1ccccc1C(CCNC(C)c1ccc2c(c1)OCO2)c1ccc(Cl)cc1 ; COc1ccc(C(CCNC(C)c2ccc(N(C)C)cc2)c2ccccc2OC)cc1]

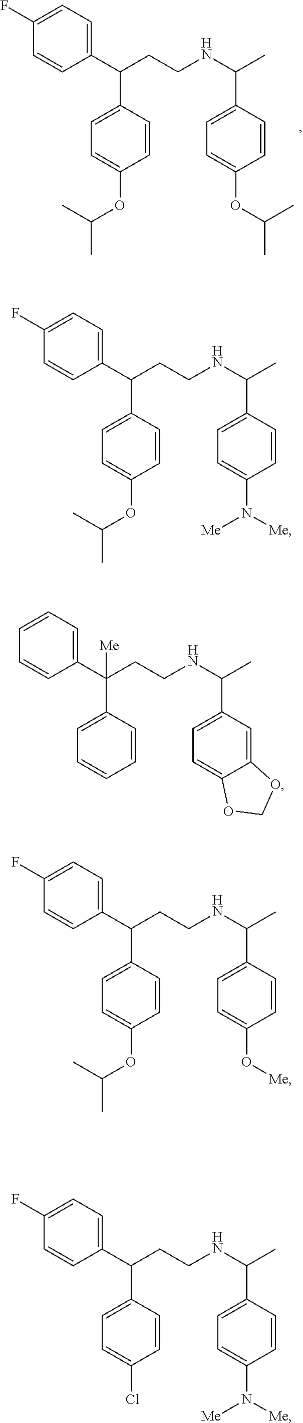 [SMILES: CC(C)Oc1ccc(C(C)NCCC(c2ccc(F)cc2)c2ccc(OC(C)C)cc2)cc1 ; CC(C)Oc1ccc(C(CCNC(C)c2ccc(N(C)C)cc2)c2ccc(F)cc2)cc1 ; CC(NCCC(C)(c1ccccc1)c1ccccc1)c1ccc2c(c1)OCO2 ; COc1ccc(C(C)NCCC(c2ccc(F)cc2)c2ccc(OC(C)C)cc2)cc1 ; CC(NCCC(c1ccc(F)cc1)c1ccc(Cl)cc1)c1ccc(N(C)C)cc1]

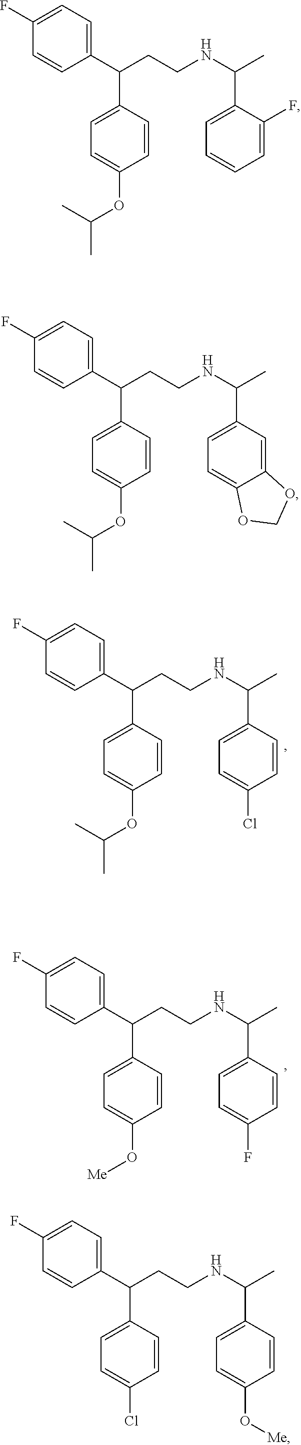 [SMILES: CC(C)Oc1ccc(C(CCNC(C)c2ccccc2F)c2ccc(F)cc2)cc1 ; CC(C)Oc1ccc(C(CCNC(C)c2ccc3c(c2)OCO3)c2ccc(F)cc2)cc1 ; CC(C)Oc1ccc(C(CCNC(C)c2ccc(Cl)cc2)c2ccc(F)cc2)cc1 ; COc1ccc(C(CCNC(C)c2ccc(F)cc2)c2ccc(F)cc2)cc1 ; COc1ccc(C(C)NCCC(c2ccc(F)cc2)c2ccc(Cl)cc2)cc1]

or a pharmaceutically acceptable salt and/or solvate thereof.

In any embodiment herein, it may be that the compound is of Formula IC

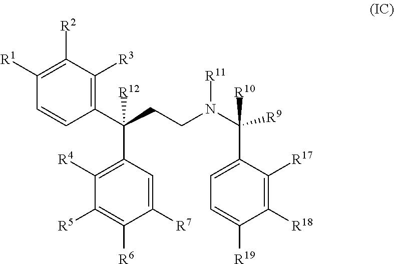

or a pharmaceutically acceptable salt and/or solvate thereof.

In any embodiment herein, it may be that the compound of Formula I is

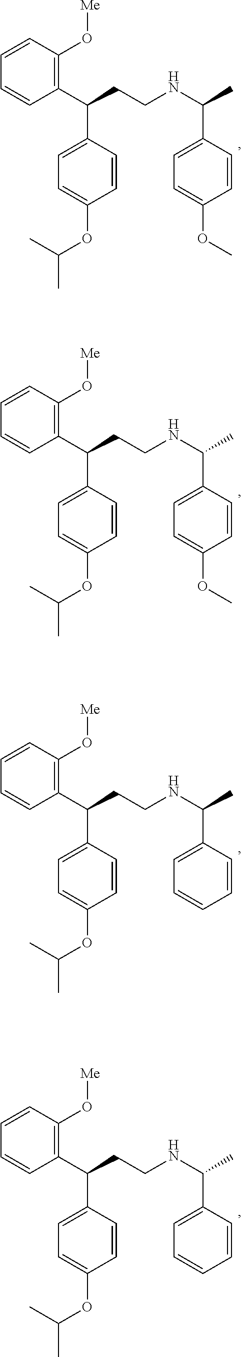 [SMILES: COc1ccc([C@H](C)NCC[C@@H](c2ccc(OC(C)C)cc2)c2ccccc2OC)cc1 ; COc1ccc([C@@H](C)NCC[C@@H](c2ccc(OC(C)C)cc2)c2ccccc2OC)cc1 ; COc1ccccc1[C@@H](CCN[C@@H](C)c1ccccc1)c1ccc(OC(C)C)cc1 ; COc1ccccc1[C@@H](CCN[C@H](C)c1ccccc1)c1ccc(OC(C)C)cc1]

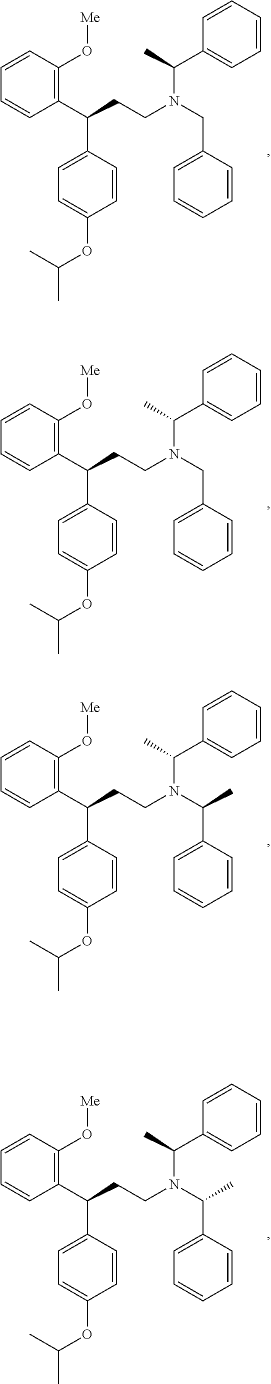 [SMILES: COc1ccccc1[C@@H](CCN(Cc1ccccc1)[C@@H](C)c1ccccc1)c1ccc(OC(C)C)cc1 ; COc1ccccc1[C@@H](CCN(Cc1ccccc1)[C@H](C)c1ccccc1)c1ccc(OC(C)C)cc1 ; COc1ccccc1[C@@H](CCN([C@H](C)c1ccccc1)[C@@H](C)c1ccccc1)c1ccc(OC(C)C)cc1 ; COc1ccccc1[C@@H](CCN([C@H](C)c1ccccc1)[C@@H](C)c1ccccc1)c1ccc(OC(C)C)cc1]

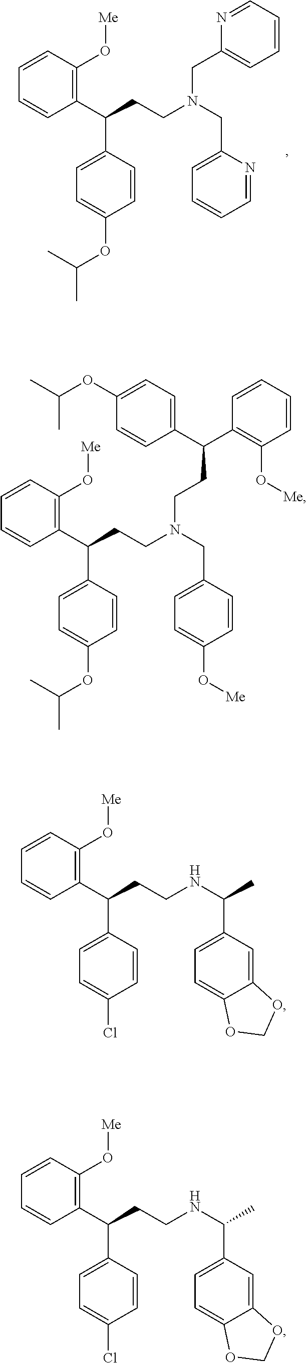 [SMILES: COc1ccccc1[C@@H](CCN(Cc1ccccn1)Cc1ccccn1)c1ccc(OC(C)C)cc1 ; COc1ccc(CN(CC[C@@H](c2ccc(OC(C)C)cc2)c2ccccc2OC)CC[C@@H](c2ccc(OC(C)C)cc2)c2ccccc2OC)cc1 ; COc1ccccc1[C@@H](CCN[C@@H](C)c1ccc2c(c1)OCO2)c1ccc(Cl)cc1 ; COc1ccccc1[C@@H](CCN[C@H](C)c1ccc2c(c1)OCO2)c1ccc(Cl)cc1]

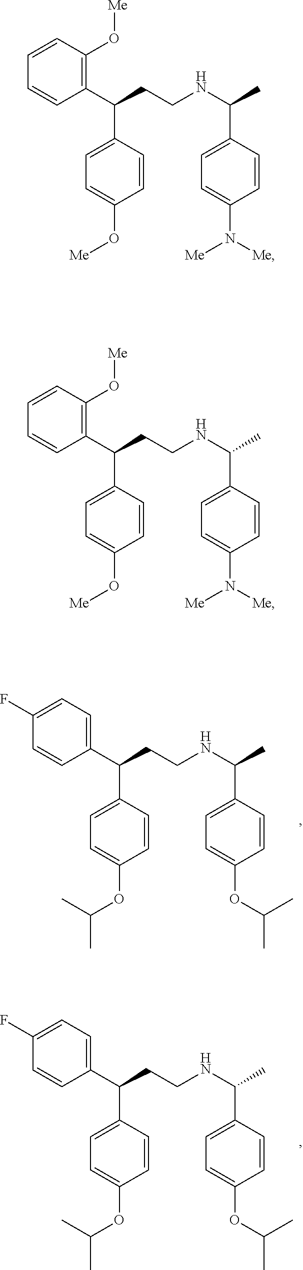 [SMILES: COc1ccc([C@H](CCN[C@@H](C)c2ccc(N(C)C)cc2)c2ccccc2OC)cc1 ; COc1ccc([C@H](CCN[C@H](C)c2ccc(N(C)C)cc2)c2ccccc2OC)cc1 ; CC(C)Oc1ccc([C@H](CCN[C@@H](C)c2ccc(OC(C)C)cc2)c2ccc(F)cc2)cc1 ; CC(C)Oc1ccc([C@H](CCN[C@H](C)c2ccc(OC(C)C)cc2)c2ccc(F)cc2)cc1]

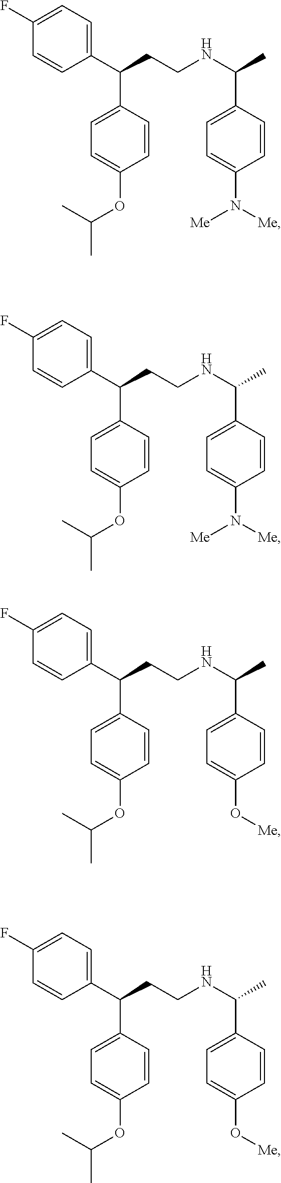 [SMILES: CC(C)Oc1ccc([C@H](CCN[C@@H](C)c2ccc(N(C)C)cc2)c2ccc(F)cc2)cc1 ; CC(C)Oc1ccc([C@H](CCN[C@H](C)c2ccc(N(C)C)cc2)c2ccc(F)cc2)cc1 ; COc1ccc([C@H](C)NCC[C@H](c2ccc(F)cc2)c2ccc(OC(C)C)cc2)cc1 ; COc1ccc([C@@H](C)NCC[C@H](c2ccc(F)cc2)c2ccc(OC(C)C)cc2)cc1]

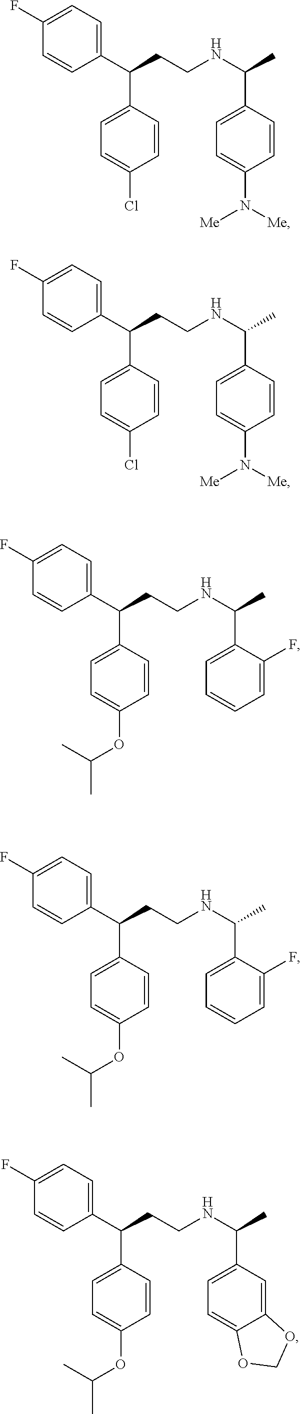 [SMILES: C[C@H](NCC[C@H](c1ccc(F)cc1)c1ccc(Cl)cc1)c1ccc(N(C)C)cc1 ; C[C@@H](NCC[C@H](c1ccc(F)cc1)c1ccc(Cl)cc1)c1ccc(N(C)C)cc1 ; CC(C)Oc1ccc([C@H](CCN[C@@H](C)c2ccccc2F)c2ccc(F)cc2)cc1 ; CC(C)Oc1ccc([C@H](CCN[C@H](C)c2ccccc2F)c2ccc(F)cc2)cc1 ; CC(C)Oc1ccc([C@H](CCN[C@@H](C)c2ccc3c(c2)OCO3)c2ccc(F)cc2)cc1]

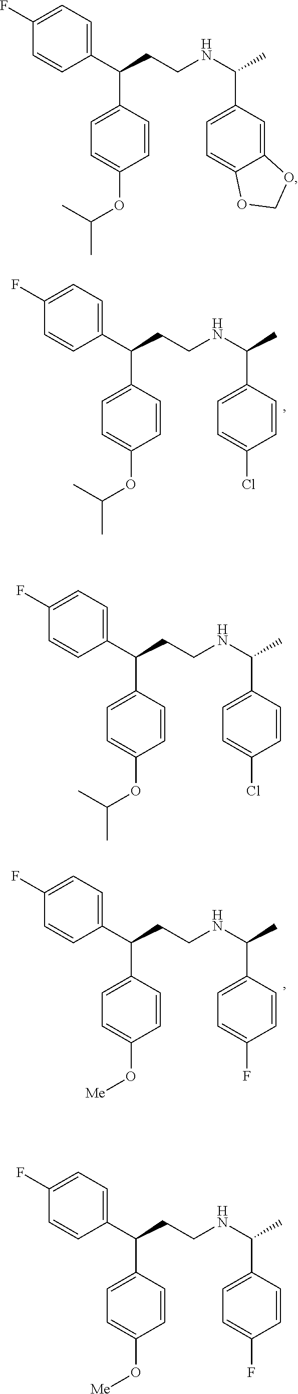

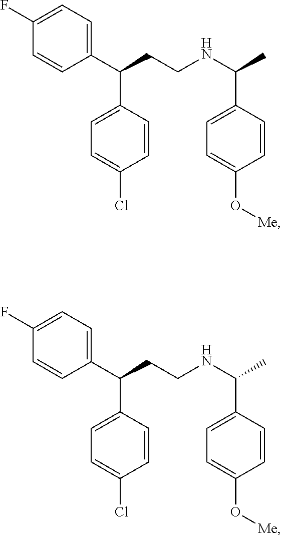 [SMILES: COc1ccc([C@H](C)NCC[C@H](c2ccc(F)cc2)c2ccc(Cl)cc2)cc1 ; COc1ccc([C@@H](C)NCC[C@H](c2ccc(F)cc2)c2ccc(Cl)cc2)cc1]

or a pharmaceutically acceptable salt and/or solvate thereof.

In any embodiment herein, the compound of Formula I may be

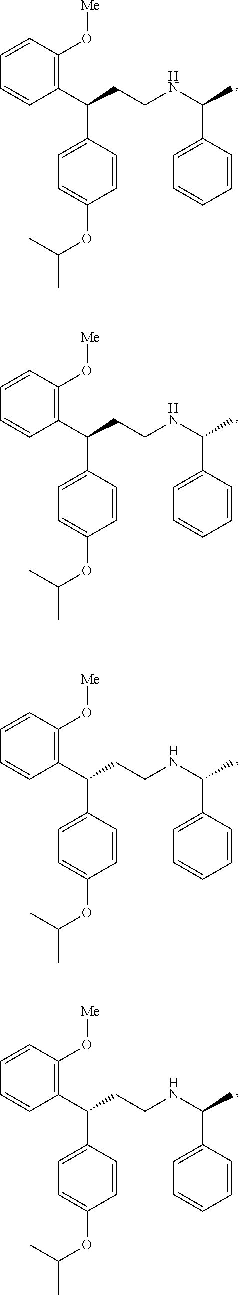

or a pharmaceutically acceptable salt and/or solvate thereof.

In an aspect, a composition is provided that includes a compound of Formula I of any embodiment disclosed herein (e.g., a compound according to Formula I, a compound disclosed above, a pharmaceutically acceptable salt and/or solvate of any compound disclosed above) and a pharmaceutically acceptable carrier or one or more excipients, fillers or agents (collectively referred to hereafter as “pharmaceutically acceptable carrier” unless otherwise indicated and/or specified). In a related aspect, a medicament is provided that includes a compound of Formula I of any embodiment disclosed herein. In a related aspect, a pharmaceutical composition is provided that includes (i) an effective amount of a compound of Formula I of any embodiment disclosed herein, and (ii) a pharmaceutically acceptable carrier. For ease of reference, the compositions, medicaments, and pharmaceutical compositions of the present technology may collectively be referred to herein as “compositions.” In further related aspects, the present technology provides methods including a compound of any embodiment disclosed herein and/or a composition of any embodiment disclosed herein as well as uses of a compound of any embodiment disclosed herein and/or a composition of any embodiment disclosed herein. Such methods and uses may include an effective amount of a compound of any embodiment disclosed herein. In any aspect or embodiment disclosed herein (collectively referred to herein as “any embodiment herein,” “any embodiment disclosed herein,” or the like), the effective amount may be an amount that treats pathogenic cellular proliferation, angiogenesis, cancer, metastatic disease, and/or a pathogenic vascular proliferative disease in a subject. As used herein, a “subject” or “patient” is typically a mammal, such as a cat, dog, rodent or primate. Typically the subject is a human, and, preferably, a human suffering from or suspected of suffering from pathogenic cellular proliferation, angiogenesis, cancer, metastatic disease, and/or a pathogenic vascular proliferative disease. The term “subject” and “patient” can be used interchangeably.

Thus, the instant present technology provides pharmaceutical compositions and medicaments comprising any of the compounds disclosed herein of the present technology (e.g., any embodiment disclosed herein of a compound of Formula I) and a pharmaceutically acceptable carrier. The compositions may be used in the methods and treatments described herein. Such compositions and medicaments include a therapeutically effective amount of any compound as described herein, including but not limited to a compound of Formula I. The pharmaceutical composition may be packaged in unit dosage form. The unit dosage form is effective in treating pathogenic cellular proliferation, angiogenesis, cancer, metastatic disease, and/or a pathogenic vascular proliferative disease when administered to a subject in need thereof.

The pharmaceutical compositions and medicaments of the present technology may be prepared by mixing one or more compounds of the present technology, pharmaceutically acceptable salts thereof, stereoisomers thereof, tautomers thereof, or solvates thereof, with pharmaceutically acceptable carriers, excipients, binders, diluents or the like. The compounds and compositions described herein may be used to prepare formulations and medicaments that prevent or treat pathogenic cellular proliferation, angiogenesis, cancer, metastatic disease, and/or a pathogenic vascular proliferative disease. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until a desired result is achieved. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition, and the pharmacological activity of the particular compound being used. The determination of optimum dosages for a particular patient is well known to those skilled in the art.

Various assays and model systems can be readily employed to determine the therapeutic effectiveness of the treatment according to the present technology. Effectiveness of the compositions and methods of the present technology may also be demonstrated by a decrease in symptoms.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

For example, the effectiveness of compounds, compositions, and method of the present technology against cancer and metastatic disease may be monitored in terms of clinical success by any of a variety of methods, for example by tumor imaging with x-rays or MRIs (e.g., to determine if tumors have decreased in size or number in treated patients). Effectiveness may often be determined by radiographic or MRI observation of a decrease in tumor size. Effective compounds, compositions, and method of the present technology for treating cancer will routinely yield at least a 10%, 25%, 50%, 75%, 90% or greater reduction of tumor size in treated patients, or average tumor size among a group of treated patients, compared to qualified, comparable control subjects. Effectiveness of compounds, compositions, and method of the present technology directed against cancer and metastatic disease may further be determined by measuring the number of circulating tumor cells in blood samples between suitable test and control subjects. This may be accomplished by any means applicable including, but not limited to immunomagnetic selection, flow cytometry, immunobead capture, fluorescence microscopy, cytomorphologic analysis, or cell separation technology. Effective compounds, compositions, and method of the present technology for treating cancer will routinely yield at least a 10%, 25%, 50%, 75%, 90% or greater reduction of circulating tumor cells in blood samples of treated patients, or among a group of treated patients, compared to qualified, comparable control subjects. Effectiveness of compounds, compositions, and method of the present technology directed against cancer and metastatic disease may further may also be determined by detecting or measuring primary tumor cell occurrence or number in secondary tissues or organs, including but not limited to bone, lymph nodes and lung. Effective compounds, compositions, and method of the present technology for treating cancer will routinely yield at least a 10%, 25%, 50%, 75%, 90% or greater reduction in the occurrence or number of primary tumor cells metastasized to secondary tissues or organs among treated patients compared to qualified, comparable control subjects.

In any embodiment or aspect herein, anti-angiogenic compounds, compositions, and method of the present technology may be effective to reduce pathologic ocular neovascularization in a mammalian subject. These methods may employ monotherapy or combination therapy. The compounds, compositions, and method of the present technology are “anti-angiogenic effective”, for example, to reduce incidence, size, or number of vascular lesions in an ocular tissue of a subject presenting with age-related macular degeneration (AMD). “Reducing neovascularization” may correspond to an observed reduction in a histopathologic or ocular angiography index of AMD lesion size, for example a reduced occurrence, size, number or distribution of lesions or “foci” of lesions observed at a secondary ocular site. Anti-angiogenic efficacy may be determined by a positive change in one or more patient therapeutic indices correlating with effective prevention and/or treatment of AMDs, e.g., by an increase in a time period of disease free or disease stable conditions for subjects receiving the a compound/composition of the present technology compared to suitable control subjects not receiving the compound/composition of the present technology.

Anti-AMD lesion efficacy, e.g., efficacy diminishing or stabilizing growth of the neovascular lesion complex, of the compounds, compositions, and methods of the of the present technology may yield substantial therapeutic benefits and improved treatment outcomes in patients treated for an ocular condition (or any other pathogenic condition) with harmful angiogenesis as part of its underlying pathology. For example, patients treated with the compounds, compositions, and methods of the present technology may exhibit improved treatment outcomes with no increase or an observed decrease in adverse side effects. Illustrative of these benefits, compounds, compositions, and methods of the present technology may yield at least a 20% increase in one or more positive clinical therapeutic indices for example a beneficial change in AMD lesion index (eg, a reduction in occurrence, size, number or distribution of the lesion or “foci” of the primary lesion observed at a secondary ocular site. Anti-AMD lesion efficacy of the compounds, compositions, and method of the present technology may be demonstrable indirectly by at least a 20% increase in a disease-free or disease stable condition for patients treated with compounds/compositions of the present technology compared to survival determined in suitable control patients (not treated with a compound/composition of the present technology). Compounds, compositions, and methods of the present technology may result in even greater anti-AMD clinical benefit, for example yielding a 20-50% increase in a positive therapeutic index, 50-90% increase, up to a 75%-100% increase, including total remission of observed primary AMD lesion enduring for 6 months to a year, 1-2 years, 2-5 years, 5 years or greater, including 10 year and longer remission. Compounds, compositions, and method of the present technology may be anti-AMD effective to yield at least a 20% decrease in lesion size, a 20%-50%, a 50%-75%, up to a 90% or greater decrease in lesion size, e.g., as demonstrated by comparative histopathology, ocular angiography, optical coherence tomography (OCT) or another ocular imaging technique in subjects treated with a compound/composition of the present technology versus non-treated or placebo-treated subjects. Anti-AMD efficacy may correlate with no increase or even a decrease in observed symptoms of AMD, e.g., loss of visual acuity between patients treated with compounds/compositions of the present technology and positive control-treated subjects. For example, subjects including subjects treated with monotherapy via the compounds/compositions of the present technology, and subjects treated with combinatorial methods such as compounds/compositions of the present technology plus anti-VEGF therapy may exhibit no increase in Snellen chart score and may exhibit at least a 20% increase, a 20-50% increase, up to a 50-90% or greater increase in Snellen chart score compared to positive control subjects treated with conventional (e.g., anti-VEGF) therapy.

The compounds of the present technology may also be administered to a patient along with other conventional therapeutic agents that may be useful in the treatment of pathogenic cellular proliferation, angiogenesis, cancer, metastatic disease, and/or a pathogenic vascular proliferative disease. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment of pathogenic cellular proliferation, angiogenesis, cancer, metastatic disease, and/or a pathogenic vascular proliferative disease.

In an aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest including, but not limited to, a cell or tissue associated with pathogenic cellular proliferation, angiogenesis, cancer, metastatic disease, and/or a pathogenic vascular proliferative disease. Suitable labels are known by those skilled in the art and can include, for example, radioisotopes, radionuclides, isotopes, fluorescent groups, biotin (in conjunction with streptavidin complexation), and chemoluminescent groups. Upon binding of the labeled compound to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

### Combination Therapy

As indicated previously in this disclosure, in any embodiment or aspect herein, a compound, composition, or pharmaceutical composition of any embodiment of the present technology may be combined with one or more additional therapies for the prevention or treatment of pathogenic cellular proliferation, angiogenesis, cancer, metastatic disease, and/or a pathogenic vascular proliferative disease. Additional therapeutic agents include, but are not limited to, chemotherapeutic agents, immunotherapeutic agents, surgery, radiation therapy, anti-angiogenic agents, non-steroidal anti-inflammatory drugs, or any combination thereof.

Additionally or alternatively, in any of the embodiments disclosed herein, the additional therapeutic agent may be selected from the group consisting of alkylating agents, topoisomerase inhibitors, endoplasmic reticulum stress inducing agents, antimetabolites, immunotherapeutic agents, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, endocrine/hormonal agents, bisphosphonate therapy agents, phenphormin, anti-angiogenic agents, Histone deacetylase inhibitors, and non-steroidal anti-inflammatory drugs (NSAIDs).

Additionally or alternatively, in any of the embodiments disclosed herein, the additional therapeutic agent may be a chemotherapeutic agent selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, ABRAXANE® (albumin-bound paclitaxel), protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-895 if, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and mixtures thereof.

Additionally or alternatively, in some embodiments, the additional therapeutic agent may be an antimetabolite selected from the group consisting of 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Additionally or alternatively, in some embodiments, the additional therapeutic agent may be a taxane selected from the group consisting of accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Additionally or alternatively, in some embodiments, the additional therapeutic agent may be a DNA alkylating agent selected from the group consisting of cyclophosphamide, chlorambucil, melphalan, bendamustine, uramustine, estramustine, carmustine, lomustine, nimustine, ranimustine, streptozotocin; busulfan, mannosulfan, and mixtures thereof.

Additionally or alternatively, in some embodiments, the additional therapeutic agent may be a topoisomerase I inhibitor selected from the group consisting of SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-895 if, MAG-CPT, and mixtures thereof.

Additionally or alternatively, in some embodiments, the additional therapeutic agent may be a topoisomerase II inhibitor selected from the group consisting of amsacrine, etoposide, etoposide phosphate, teniposide, daunorubicin, mitoxantrone, amsacrine, ellipticines, aurintricarboxylic acid, doxorubicin, and HU-331 and combinations thereof.

Additionally or alternatively, in some embodiments, the additional therapeutic agent may be an immunotherapeutic agent selected from the group consisting of immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

Additionally or alternatively, in some embodiments, the additional therapeutic agent may be an anti-angiogenic agent selected from the group consisting of bevacizumab, cediranib, axitinib, anginex, sunitinib, sorafenib, pazopanib, vatalanib, cabozantinib, ponatinib, lenvatinib, SU6668, Everolimus (Afinitor®), Lenalidomide (Revlimid®), Ramucirumab (Cyramza®), Regorafenib (Stivarga®), Thalidomide (Synovir, Thalomid®), Vandetanib (Caprelsa®), and Ziv-aflibercept (Zaltrap®).

Additionally or alternatively, in some embodiments, the additional therapeutic agent may be a histone deacetylase inhibitor selected from the group consisting of trichostatin A (TSA), tubacin, apicidin, depsipeptide, MS275, BML-210, RGFP966, MGCD0103, LBH589, splitomicin, FK₂₂₈, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC202, Mocetinostat, Romidepsin, Nicotinamide, Sirtinol, Cambinol, and EX-527.

Additionally or alternatively, in some embodiments, the additional therapeutic agent may be a NSAID selected from the group consisting of indomethacin, fenoprofen, ibuprofen, flufenamic acid, aspirin, celecoxib, diclofenac, diflunisal, etodolac, ketoprofen, ketorolac, nabumetone, naproxen, oxaprozin, piroxicam, salsalate, sulindac, and tolmetin.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of immunotherapeutic agents include immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

Examples of anti-angiogenic agents include bevacizumab, cediranib, axitinib, anginex, sunitinib, sorafenib, pazopanib, vatalanib, cabozantinib, ponatinib, lenvatinib, SU6668, Everolimus (Afinitor®), Lenalidomide (Revlimid®), Ramucirumab (Cyramza®), Regorafenib (Stivarga®), Thalidomide (Synovir, Thalomid®), Vandetanib (Caprelsa®), and Ziv-aflibercept (Zaltrap®).

Examples of histone deacetylase inhibitors include trichostatin A (TSA), tubacin, apicidin, depsipeptide, MS275, BML-210, RGFP966, MGCD0103, LBH589, splitomicin, FK₂₂₈, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC202, Mocetinostat, Romidepsin, Nicotinamide, Sirtinol, Cambinol, and EX-527.

Examples of NSAIDs include indomethacin, fenoprofen, ibuprofen, flufenamic acid, aspirin, celecoxib, diclofenac, diflunisal, etodolac, ketoprofen, ketorolac, nabumetone, naproxen, oxaprozin, piroxicam, salsalate, sulindac, and tolmetin.

In any case, in any embodiment herein, the multiple therapeutic agents may be administered in any order or even simultaneously. If simultaneously, the multiple therapeutic agents may be provided in a single, unified form, or in multiple forms (by way of example only, either as a single injection, as two separate injections, or as an injection in combination with pill). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

The present disclosure also provides kits comprising compounds and/or compositions of the present technology and instructions for using the same to prevent and/or treat pathogenic cellular proliferation, angiogenesis, cancer, metastatic disease, and/or a pathogenic vascular proliferative disease. Optionally, the above described components of the kits of the present technology are packed in suitable containers and labeled for the prevention and/or treatment of pathogenic cellular proliferation, angiogenesis, cancer, metastatic disease, and/or a pathogenic vascular proliferative disease.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable carrier of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology. In any embodiment herein, the written product may instruct performance of a method according to any embodiment described herein.

### Examples

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology. The following Examples demonstrate the preparation, characterization, and use of illustrative compositions of the present technology that inhibit Id proteins.

### Synthesis of Comparative Compound AGX51.

The reaction of 2-methoxycinnamaldehyde 1 and the potassium aryltrifluoroborate 2a or regular arylboronic acid 2b, provided the desired adduct 3 in high yield (Scheme 1). Reductive amination of aldehyde 3 with benzylamine and sodium borohydride afforded amine 4. Acylation of amine 4 with propionyl chloride provided the final product 5 (AGX51) in 75% overall yields.

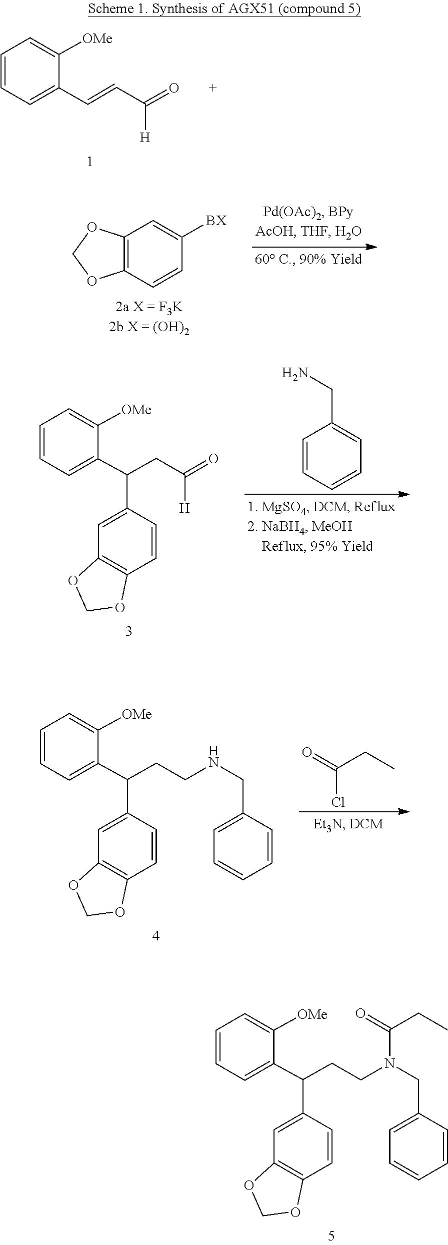

### Synthesis of Comparative Compound AGX-A.

Compound 7 was generated in one step by conjugate addition of trifluoroborate 6 to aldehyde 1 in the presence of catalytic amount of Palladium dibenzylideneacetone and triphenylphosphine. Reductive amination of aldehyde 7 gives secondary amine 8 (AGX-A) in high yield as a viscous oil which was transformed into the corresponding hydrochloride salt 9 and was isolated as a white solid.

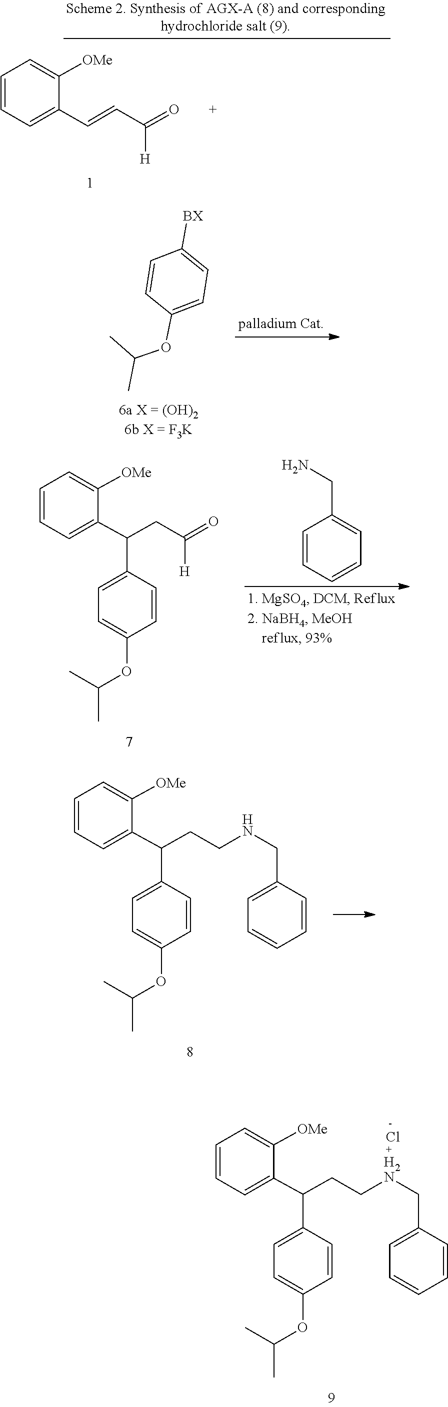

### Exemplary Reductive Amination Using Amines Bearing Carbonyl Groups.

The two-step reductive amination of aldehyde 3 (Scheme 3) with amines bearing incompatible groups such as ketones which could be reduced by reagents such as NaBH₄ were instead hydrogenated using hydrogen under palladium on charcoal catalysis. Compound 10 was obtained by hydrogenation of the intermediate imine using Pd 10% in charcoal under 1 Atmosphere hydrogen.

### Scheme 3. Synthesis of Compound 11.

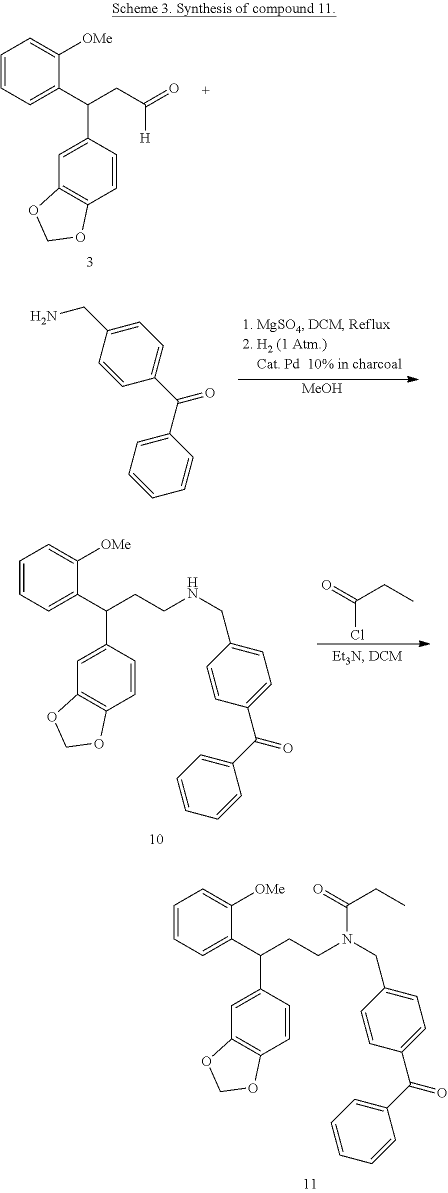

### Chiral Resolution of Comparative Compound AGX51

As indicated in Scheme 4, racemic AGX51 was resolved on a chiral AS-H prep column (Chiral technologies) to afford compound P1 (peak 1, >99% ee, [a]^{21.6}D+22.53 (c 0.8, MeOH)) and compound P2 (peak 2, ˜93% ee, [a]^{21.6}D −25.77 (c 0.8, MeOH)).

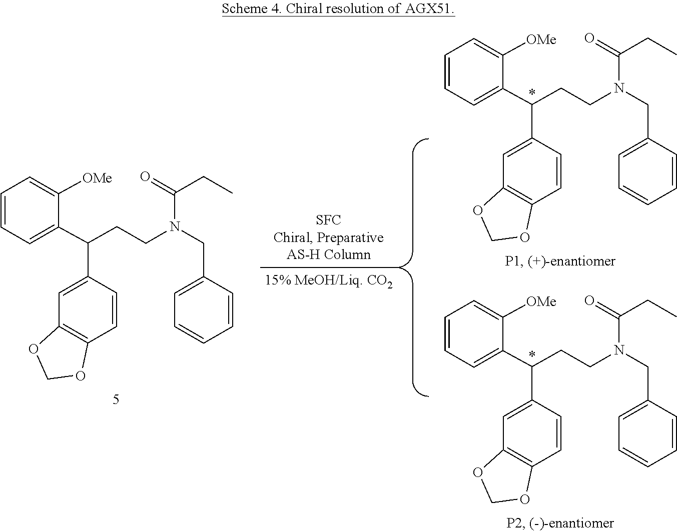 [SMILES: CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1]

Neither P1 nor P2 provided X-Ray quality crystals. However, because the corresponding amine salts were crystalline, the amide could be hydrolyzed under conditions, which should not compromise the chiral center. Hence, as indicated in Scheme 5, the (+)-enantiomer was submitted to 4.0 normal HCl in Dioxane-water at 85° C. for 24 hours which provided the corresponding amine after basic work up.

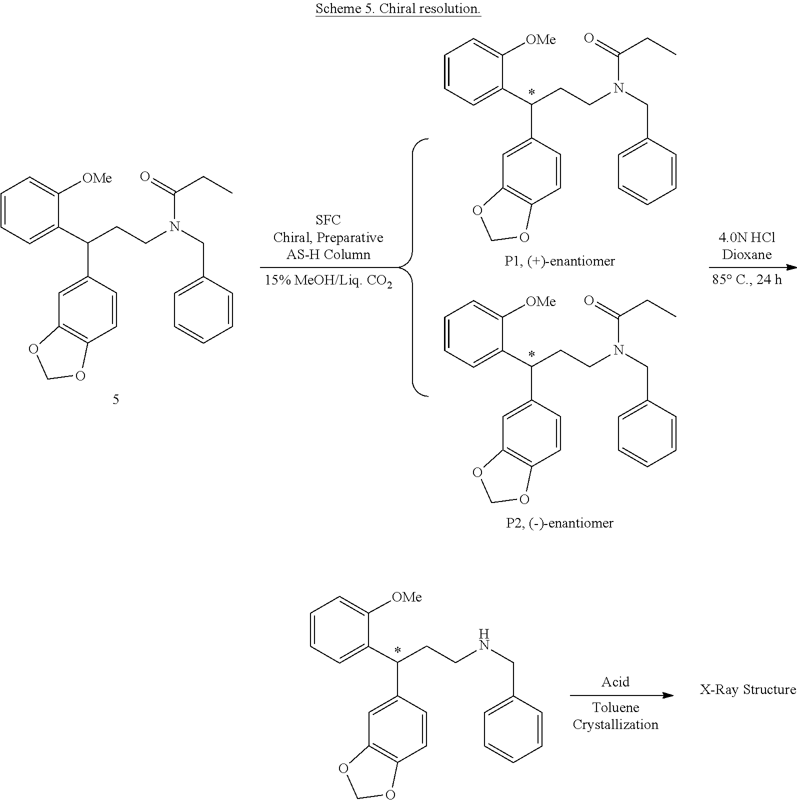 [SMILES: CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1 ; COc1ccccc1C(CCNCc1ccccc1)c1ccc2c(c1)OCO2]

Thus obtained (+)-chiral amine was then crystallized with several acids; (+)-camphor sulfonic acid, D-(−)-tartaric acid, L-(+)-tartaric acid, L-(−)-malic acid, and fumaric acid in ethanol (toluene outer chamber). Crystals were harvested and submitted for X-Ray crystallography. Only crystals from the fumarate diffracted and provided unequivocally the conformation of the chiral center as the R-(+)-enantiomer.

Deacylation of both optically pure amides P1 and P2 was additionally achieved by in situ activation of the inert tertiary amide followed by addition of phenyl Grignard reagent (Huang at al., Tetrahedron, 71(2015): 4248-4254), as indicated in Scheme 6. NMR analysis of both optical amines is identical to the one of synthesized amine 4 from Scheme 1

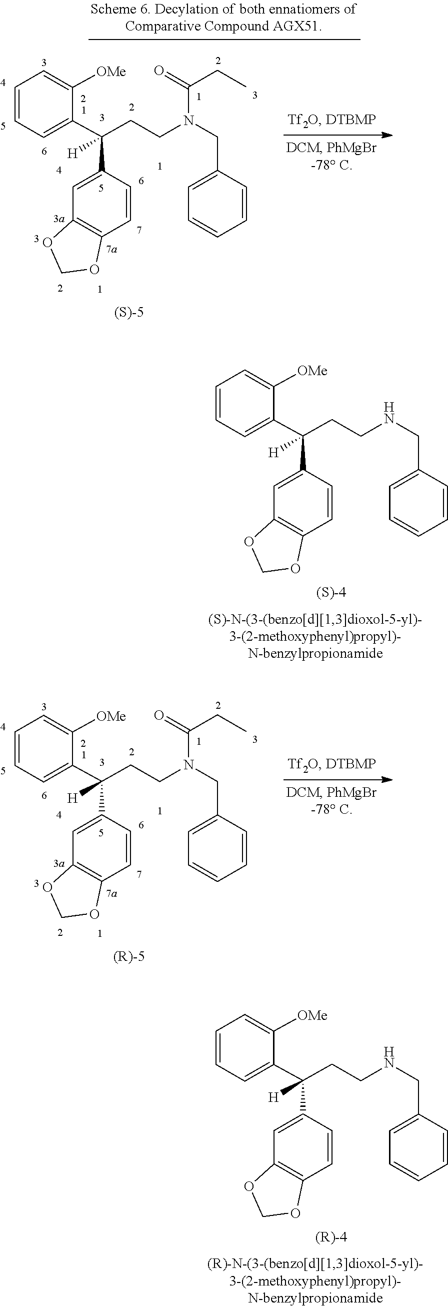 [SMILES: CCC(=O)N(CC[C@@H](c1ccc2c(c1)OCO2)c1ccccc1OC)Cc1ccccc1 ; COc1ccccc1[C@@H](CCNCc1ccccc1)c1ccc2c(c1)OCO2 ; CCC(=O)N(CC[C@H](c1ccc2c(c1)OCO2)c1ccccc1OC)Cc1ccccc1 ; COc1ccccc1[C@H](CCNCc1ccccc1)c1ccc2c(c1)OCO2]

Additionally, as indicated in Scheme 7, the direct chiral synthesis could be achieved through organo-catalysis using a chiral catalyst such as R—(C₇F₇)₂-BINOL (Angew. Chem. Int. Ed. Eng., 54(34): 9931-9935 (2015)). Indeed, reacting aldehyde 1 with trifluoroboronate 2a in the presence of catalytic amounts of R—(C₇F₇)₂-BINOL in toluene at 95° C. with molecular sieves, produced chiral aldehyde 3, which was advanced to the corresponding chiral amine 12. Following functional group manipulations to install the trifluoro acetamide and remove the Boc protecting group, reductive amination on the resulting amine using aldehyde 15 provided diazirine 16 which displayed a specific rotation of [a]²²D −70.39 (c 1.0, CH₂Cl₂). Enantiomeric excess was not determined. Aldehyde 15 was the product of a Dess-Martin periodinane oxidation of alcohol 14.

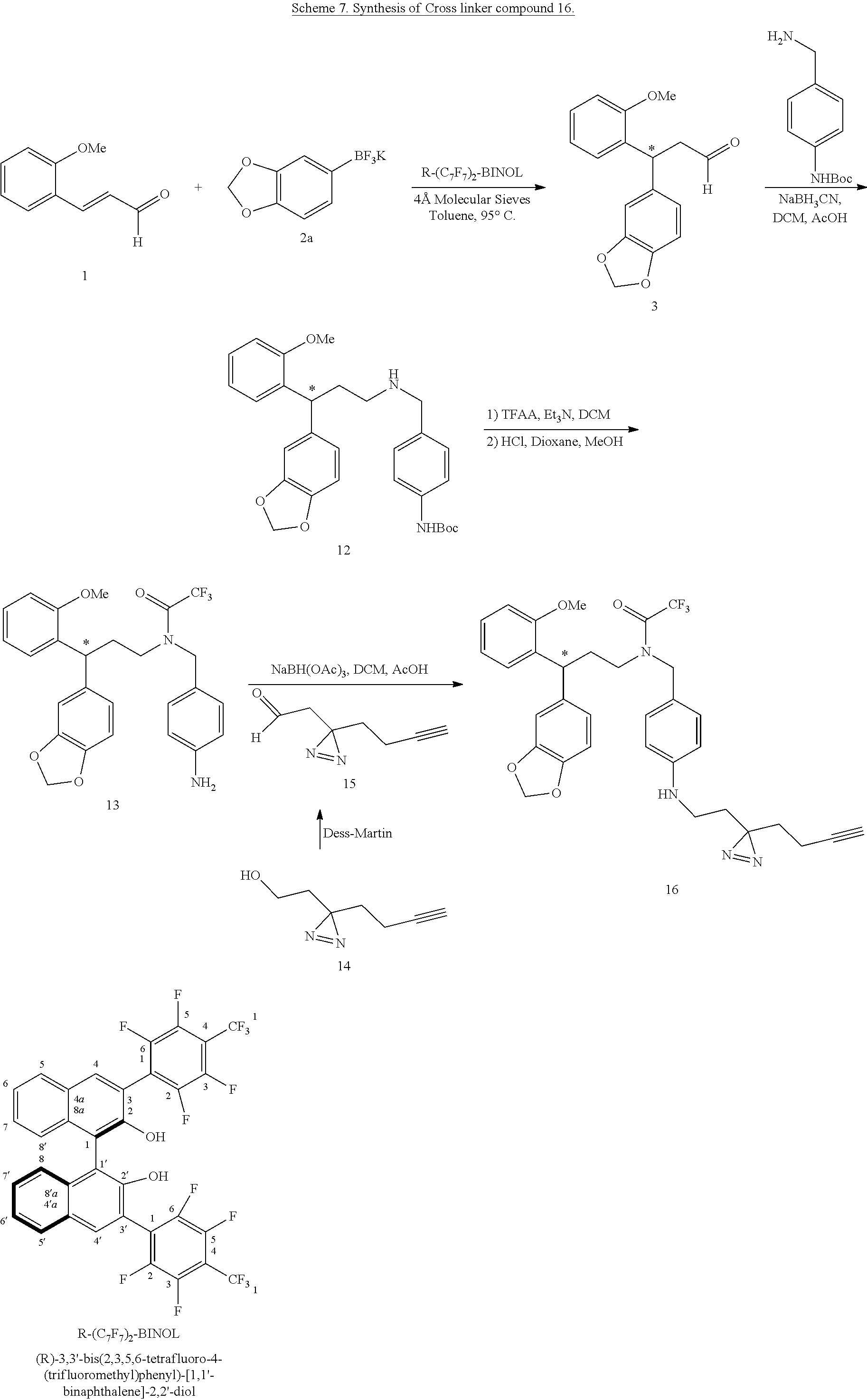

### Exemplary Synthetic Procedures

Typical procedure A for conjugate addition of potassium organotrifluoroborate 2a to 2-methoxycinnamaldehyde. To a flask were added potassium organotrifluoroborate 2a (6.183 g, 27 mmol, 2.5 equ), 2-methoxycinnamaldehyde 1 (1.76 g, 10.85 mmol), Pd(OAc)₂ (122 mg, 0.54 mmol, 5 mol %), bpy (339 mg, 2.17 mmol, 20 mol %), HOAc (10 mL), THF (5.4 mL), and H₂O (3.2 mL) under Argon. The mixture was stirred and heated at 60° C. for 2-3 days. The reaction mixture was cooled to room temperature and filter and washed with EtOAc. The filtrate was neutralized with saturated NaHCO₃ and then extracted with ethyl acetate (×3). The combined organic layers was washed with saturated NaCl, dried (Na₂SO₄) and concentrated. The residue was purified by ISCO Combi Flash SiO₂ (12 g) column (20% ethyl acetate/cyclohexane) to give the product 3 (2.83 g) with 92% yield as a yellow oil.

Typical procedure B for conjugate addition of Arylboronic acid to 2-methoxycinnamaldehyde. To a flask were added arylboronic acid 6a (2.82 g, 15.67 mmol, 2.5 eq), 2-methoxycinnamaldehyde 1 (1 g, 6.17 mmol), Pd(OAc)₂ (69 mg, 0.308 mmol, 5 mol %), bpy (192 mg, 1.22 mmol, 20 mol %), HOAc (6 mL), THF (3 mL), and H₂O (1.8 mL) under Argon. The mixture was stirred and heated at 60° C. for 2-3 days. The reaction mixture was neutralized with saturated NaHCO₃ and then extracted with ethyl acetate (×3). The combined organic layers was washed with saturated NaCl, dried (Na₂SO₄) and concentrated. The residue was purified by ISCO Combi Flash SiO₂ (24 g) column (25% ethyl acetate/cyclohexane) to give the product 7 (1.46 g) with 80% yield as a yellow oil.

General procedure C Reductive amination: To a solution of aldehyde 3 (7.66 g, 26.96 mmol) and benzyl amine (3.17 g, 29.66 mmol) in dichloromethane (150 mL) was added anhydrous magnesium sulfate (4.85 g, 40.44 mmol). After stirring for 1 h at reflux, the reaction mixture was filtered to remove the drying agent and the solvent was removed in vacuo. The crude imine was then dissolved in methanol (100 mL) and sodium borohydride (2.04 g, 53.92 mmol) was added while stirring at 0° C. After additional stirring for 1 h at reflux, the reaction mixture was quenched with water and concentrated to remove methanol. The residue was diluted with dichloromethane, and washed with water. The aqueous layer was extracted with dichloromethane (×3), the combined organic layer was washed saturated NaCl, dried (Na₂SO₄) and concentrated. The residue was purified by ISCO Combi Flash SiO₂ (120 g) column (5% MeOH/ethyl acetate) to give the product 5 (9.36 g) with 92% yield as a yellow oil.

3-(benzo[d][1,3]dioxol-5-yl)-3-(2-methoxyphenyl)propanal 3. ¹H NMR (CDCl, 600 MHz) S 9.69 (t, J=2.2 Hz, 1H), 7.19 (dt, J=7.5, 1.6 Hz, 1H), 7.07 (dd, J=7.6, 1.5 Hz, 1H), 6.9 (t, J=7.5 Hz, 1H), 6.86 (d, J=8.2 Hz, 1H), 6.73 (m, 3H), 5.91 (s, 2H), 4.95 (t, J=7.9 Hz, 1H), 3.82 (s, 3H), 3.05 (dd, J=7.9, 2.2 Hz, 2H); ¹³C NMR (CDCl₃, 150 MHz) δ 201.75, 156.53, 147.71, 146.06, 136.75, 131.65, 127.88, 127.84, 120.88, 120.71, 110.78, 108.66, 108.14, 100.92, 55.40, 48.63, 37.96.

N-benzyl-3-(4-isopropoxyphenyl)-3-(2-methoxyphenyl)propan-1-amine 4 ¹H NMR (CDCl₃, 600 MHz) δ 7.30-7.19 (m, 6H), 7.15 (dt, J=8.1, 1.4 Hz, 1H), 6.89 (t, J=7.4 Hz, 1H), 6.81 (d, J=8.1 Hz, 1H), 6.73 (m, 2H), 6.68 (d, J=8.0 Hz, 1H), 5.86 (s, 2H), 4.41 (t, J=7.8 Hz, 1H), 3.76 (s, 3H), 3.72 (s, 2H), 2.60 (t, J=7.1 Hz, 2H), 2.17 (m, 2H); ¹³C NMR (CDCl₃, 150 MHz) δ 156.99, 147.58, 145.72, 140.67, 138.46, 133.46, 128.51, 128.23, 127.66, 127.32, 127.00, 121.10, 120.82, 110.84, 108.80, 108.10, 100.89, 55.61, 54.03, 47.95, 40.74, 35.45.

N-(3-(benzo[d][1,3]dioxol-5-yl)-3-(2-methoxyphenyl)propyl)-N-benzylpropionamide 5 (AGX51)

General procedure D: To a solution of the amine 8 (6.14 g, 16.37 mmol) and triethylamine (4.56 mL, 32.75 mmol) in anhydrous dichloromethane (100 mL), propionyl chloride (3.57 mL, 40.92 mmol) was added dropwise at 0° C. After the reaction mixture was left overnight at room temperature, the resulting solution was poured into water and separated. The water layer was extracted with dichloromethane (×3). The combined organic layers were washed with saturated NaCl and then dried over (Na₂SO₄) and concentrated. The residue was purified by ISCO Combi Flash SiO₂ (120 g) column (30-40% ethyl acetate/hexanes) to give the product 10 (6.35 g) with 90% yield as a sticky yellow oil. The resulting clear sticky syrup was treated with ethanol (9 mL) and mixed well. After sitting in freezer overnight, white precipitate was formed, the white solid was filtered and washed with cold ethanol to afford dried white powder 5.77 g (AGX51). Mp: 87-88° C.

¹H NMR (DMSO-d₆, 600 MHz) δ 7.30-7.18 (m, 4H), 7.15 (m, 1H), 7.08 (m, 2H), 6.92-6.88 (m, 2H), 6.84-6.74 (m, 2H), 6.68 (m, 1H), 5.93 (m, 2H), 4.54-4.40 (m, 2H), 4.18 (t, J=7.9 Hz, 1H), 3.73, 3.72 (s, s, 3H), 3.16-2.98 (m, 2H), 2.54-2.05 (m, 4H), 0.95 (t, J=7.3 Hz, 3H); ¹³C NMR (CDCl₃, 150 MHz) δ 173.91, 173.83, 156.68, 147.66, 147.41, 145.91, 145.59, 138.34, 137.96, 137.47, 137.21, 132.79, 132.26, 128.80, 128.45, 128.24, 127.58, 127.44, 127.33, 127.19, 127.16, 126.34, 120.83, 120.81, 120.72, 120.68, 110.72, 110.66, 108.56, 108.48, 108.11, 107.95, 100.87, 100.70, 55.41, 55.37, 51.48, 48.05, 45.99, 45.36, 40.90, 40.50, 33.61, 32.54, 26.55, 26.02, 9.67, 9.48; high res MS calc. for C₂₇H₃₀NO₂₄ 432.2175, found 432.2191 (M+H).

3-(4-isopropoxyphenyl)-3-(2-methoxyphenyl)propanal 7. ¹H NMR (CDCl₃, 600 MHz) δ 9.69 (t, J=2.3 Hz, 1H), 7.18 (dt, 1H, J=8.1, 1.7 Hz), 7.14 (m, 2H), 7.05 (dd, J=7.6, 1.6 Hz, 1H), 6.88 (dt, J=7.5, 0.9 Hz, 1H), 6.85 (d, J=8.2, 0.9 Hz, 1H), 6.80 (m, 2H), 4.96 (t, J=7.9 Hz, 1H), 4.49 (m, 1H), 3.81 (s, 3H), 3.05 (dd, J=7.9, 2.3 Hz, 2H), 1.31 (d, J=6.0 Hz, 6H); ¹³C NMR (CDCl₃, 150 MHz) δ 202.15, 156.57, 156.43, 134.55, 132.05, 129.03, 128.08, 127.68, 120.67, 115.72, 110.73, 69.79, 55.39, 48.63, 37.53, 22.10.

N-benzyl-3-(4-isopropoxyphenyl)-3-(2-methoxyphenyl)propan-1-amine 8 (AGX-A). ¹H NMR (CDCl₃, 600 MHz) δ 7.29-7.18 (m, 6H), 7.16-7.12 (m, 3H), 6.88 (dt, J=7.5, 0.9 Hz, 1H), 6.81 (d, J=8.2 Hz, 1H), 6.75 (m, 2H), 4.48-4.41 (m, 2H), 3.75 (s, 3H), 3.71 (s, 2H), 2.60 (t, J=7.2 Hz, 2H), 2.19 (m, 2H), 1.29 (d, J=6.1 Hz, 6H); ¹³C NMR (CDCl₃, 150 MHz) δ 156.90, 156.04, 140.54, 136.62, 133.71, 129.02, 128.09, 127.72, 127.00, 126.83, 120.64, 115.53, 110.68, 69.77, 55.47, 53.88, 47.91, 40.09, 35.29, 30.39, 22.18; high res MS calc. for C₂₆H₃₂NO₂390.2433, found 390.2426 (M+H).

AGX-A Hydrogen chloride salt 9 formation: Benzyl amine AGX-A 8 (9.92 g, 25.5 mmol) in dichloromethane (40 mL) was treated with HCl (2 N in ether, 14 mL, 28 mmol) in ice bath. The mixture was stirred for 2 h. After removal of solvent, the sticky syrup was treated with hexane twice and concentrated in vacuo. The syrup (5 g) was titrated with ethyl acetate (10 mL) to form white precipitate. The white solid was filtered and washed with hexane to afford AGX-A HCl 9 salt (10.6 g) as a white powder. Mp: 158-160° C.

(4-(((3-(benzo[d][1,3]dioxol-5-yl)-3-(2-methoxyphenyl)propyl)amino)methyl)phenyl) (phenyl)methanone 10. 4-(Aminomethyl)phenyl](phenyl)methanone hydrochloride (66 mg, 0.27 mmol) was pretreated with K₂CO₃ (5 equiv) in dichloromethane (10 mL) and water (2 mL). After stirring at room temperature for 2 h, the aqueous layer was extracted with dichloromethane (×3), the combined organic layer was washed with saturated NaCl and then dried over (Na₂SO₄) and concentrated to afford the colorless oil directly used for the next step.

The resulting free amine was treated with aldehyde 3 using the general procedure C to afford the imine, which was treated with 10% Pd/C (48 mg) in MeOH (4 mL) and dichloromethane (2 mL) with a hydrogen balloon for 2 h. The reaction mixture was filtered through celite and washed with mixture of MeOH and dichloromethane. After removal of the solvents, the residue was purified by ISCO Combi Flash SiO₂ (4 g) column (5% MeOH/dichloromethane) to give the product 10 (60 mg) with 50% yield as a foaming solid. ¹H NMR (CDCl₃, 500 MHz) δ 7.75 (m, 4H), 7.59 (t, J=7.4 Hz, 1H), 7.53 (d, J=7.9 Hz, 2H), 7.46 (t, J=7.6 Hz, 2H), 7.13 (m, 2H), 6.86 (t, J=7.4 Hz, 1H), 6.78 (d, J=8.1 Hz, 1H), 6.70 (m, 2H), 6.63 (d, J=8.3 Hz, 1H), 5.83 (s, 2H), 4.36 (t, J=7.5 Hz, 1H), 3.97 (s, 2H), 3.75 (s, 3H), 2.71 (t, J=7.3 Hz, 2H), 2.41 (m, 2H); ¹³C NMR (CDCl₃, 125 MHz) δ 195.06, 180.18, 170.20, 155.69, 146.56, 144.86, 136.57, 136.34, 136.29, 131.63, 130.96, 129.52, 129.04, 128.27, 127.37, 126.58, 126.48, 119.85, 119.81, 109.76, 107.51, 107.06, 99.82, 59.43, 54.49, 39.57, 20.08, 13.22.

N-(3-(benzo[d][1,3]dioxol-5-yl)-3-(2-methoxyphenyl)propyl)-N-(4-benzoylbenzyl) propionamide 13. The above amine 10 was treated with propionyl chloride (29 μL, 0.313 mmol) followed with the general procedure D to afford the compound 11 (66 mg, 98%) as a sticky syrup. ¹H NMR (CDCl₃, 600 MHz) δ 7.81-7.70 (m, 4H), 7.60 (m, 1H), 7.49 (m, 2H), 7.25-7.12 (m, 4H), 6.91 (m, 1H), 6.85-6.79 (m, 1H), 6.75-6.67 (m, 3H), 5.88-5.84 (m, 2H), 4.69-4.51 (m, 2H), 4.31-4.23 (t, t, J=7.9, 7.9 Hz, 1H), 3.78, 3.75 (s, s, 3H), 3.34, 3.17 (t, t, J=7.6, 7.6 Hz, 2H), 2.30-2.22 (m, 4H), 1.12 (t, J=7.4 Hz, 3H); ¹³C NMR (CDCl₃, 150 MHz) δ 196.35, 196.21, 174.07, 173.93, 156.68, 147.71, 147.48, 145.99, 145.67, 142.84, 142.05, 138.21, 137.64, 137.45, 137.35, 136.91, 136.54, 132.69, 132.56, 132.14, 130.68, 130.38, 130.02, 130.00, 128.36, 128.29, 127.87, 127.69, 127.31, 127.13, 126.21, 120.80, 120.78, 120.72, 110.78, 110.72, 108.53, 108.44, 108.16, 108.00, 100.92, 100.75, 60.41, 55.45, 55.42, 51.36, 48.12, 46.14, 45.81, 40.90, 40.47, 33.71, 32.58, 26.60, 26.02, 21.07, 9.66, 9.47.

Preparation of Chiral AGX51 using Chiral Column. Racemic AGX51 5 (1 g) was resolved by waters company using chiral AS-H prep column eluting with 15% MeOH/liq CO₂3.5 ml/min to get AGX51 P1 (99% ee, 460 mg syrup with [a]=22.53 (c=0.8 MeOH). AGX51 P2 (93% ee, 480 mg syrup with [a]=−25.77 (c=0.8 MeOH).

General procedure for the direct transformation of chiral tertiary amide into chiral amine. Tf₂O (1.2 equiv) was added dropwise to a cooled (−78° C.) solution of amide AGX51 (P1) and 2,6-Di-tert-butyl-4-methylpyridine (DTBMP 1.2 equiv) in CH₂Cl₂ (5 mL). The reaction was allowed warming to 0° C. over 2 h. A solution of Grignard reagent PhMaBr (1 N THF, 1.0 equiv) was added dropwise to the resultant mixture at −78° C., and the mixture was stirred at the same temperature for 2 h. The reaction mixture was then quenched with an aqueous solution of NH₄Cl. The organic layer was separated and the aqueous layer was extracted with ethyl acetate (×3). The combined organic layers were washed with brine, dried over (Na₂SO₄) and concentrated. The residue was purified by ISCO Combi Flash SiO₂ column (50-70% ethyl acetate/hexanes) to give the chiral product 4.

Amine 4P1 was afforded from AGX51 (P1), [a]²¹D 39.66 (c 1.7, MeOH). Amine 4P2 was afforded from AGX51 (P2), [a]²²D −38.49 (c 1.3, MeOH).

3-(benzo[d][1,3]dioxol-5-yl)-3-(2-methoxyphenyl)propanal (3). To a flask equipped with a stir bar was added powdered 4 Å A molecular sieves (0.7 g) and the flask was flame-dried under vacuum. The flask was cooled to rt under Argon. The aldehyde 1 (100 mg, 0.616 mmol), ligand R—(C₇F₇)₂BINOL (88 mg, 0.12 mol, 0.2 equ), potassium aryl trifluoroborate salt 2a (422 mg, 1.85 mmo, 3 equ) were added followed by anhydrous toluene (12 mL). The reaction was heated at 95° C. for 3 days. The reaction mixture was then filtered through celite and washed with ether. After concentration the residue was purified on 4 g silica gel column eluting with 5-10% acetone/hexane to afford product 3 (70 mg, 40%) and recover starting material aldehyde 1. ¹H NMR (CDCl₃, 600 MHz) δ 9.69 (t, J=2.2 Hz, 1H), 7.19 (dt, J=7.5, 1.6 hz, 1H), 7.07 (dd, J=7.6, 1.5 Hz, 1H), 6.9 (t, J=7.5 Hz, 1H), 6.86 (d, J=8.2 Hz, 1H), 6.73 (m, 3H), 5.91 (s, 2H), 4.95 (t, J=7.9 Hz, 1H), 3.82 (s, 3H), 3.05 (dd, J=7.9, 2.2 Hz, 2H); ¹³C NMR (CDCl₃, 150 MHz) δ 201.75, 156.53, 147.71, 146.06, 136.75, 131.65, 127.88, 127.84, 120.88, 120.71, 110.78, 108.66, 108.14, 100.92, 55.40, 48.63, 37.96. [a]²²D −70.39 (c 1.0, CH₂Cl₂).

tert-butyl (4-(((3-(benzo[d][1,3]dioxol-S-yl)-3-(2-methoxyphenyl)propyl)amino)methyl)phenyl)carbamate 12

General procedure. A mixture of aldehyde 3 (20 mg, 0.07 mmol), tert-butyl (4-(aminomethyl)phenyl)carbamate (18 mg, 0.081 mmol) and acetic acid (5 μL, 0.086 mmol) in ClCH₂CH₂Cl (1 ml) was stirred at rt for 30 min before adding sodium triacetoxyborohydride (37 mg, 0.175 mmol). The resulting mixture was stirred at rt for 18 h. The mixture was concentrated and dissolved in MeOH and purified on 4 g silica gel column to give product 12 (25 mg, 60%) as a colorless oil. ¹H NMR (CDCl₃, 600 MHz) δ 7.26 (m, 1H), 7.19-7.13 (m, 4H), 6.89 (t, J=7.5 Hz, 1H), 6.81 (d, J=8.0 Hz, 1H), 6.71-6.66 (m, 3H), 6.55 (brs, 1H), 6.23 (br, 2H), 5.87 (s, 2H), 4.34 (t, J=7.9 Hz, 1H), 3.75 (s, 3H), 3.73 (s, 2H), 2.61 (m, 2H), 2.23 (m, 2H), 1.51 (s, 9H); ¹³C NMR (CDCl₃, 150 MHz) δ 156.73, 147.49, 145.70, 138.05, 137.87, 132.68, 129.46, 127.75, 120.89, 120.75, 118.56, 110.71, 108.57, 108.00, 100.76, 80.57, 55.45, 51.79, 46.38, 40.57, 33.44, 28.35; [a]²²D −30.45 (c 1.25, MeOH); high res MS calc. for C₂₉H₃₅N₂O₅ 491.2546, found 491.2534 (M+H).

N-(4-aminobenzyl)-N-(3-(benzo[d][1,3]dioxol-S-yl)-3-(2-methoxyphenyl)propyl)-2,2,2-trifluoroacetamide 13. A solution of amine 12 (25 mg, 0.051 mmol) and triethylamine (21 μl, 0.153 mmol) in CH₂Cl₂ (1 mL) was treated with trifluoroacetic anhydride (18 μL, 0.127 mmol) at room temperature. After the reaction mixture was stirred for 4 h, the solution was concentrated and purified on 4 g silica gel column to give product (18 mg, 60%). ¹H NMR (CDCl₃, 600 MHz) δ 7.30 (m, 1H), 7.21-7.15 (m, 1H), 7.10 (t, J=8.9 Hz, 1H), 6.92 (dd, J=8.3, 16.7 Hz, 2H), 6.92-6.80 (m, 2H), 6.72-6.67 (m, 3H), 6.48 (d, J=19.1 Hz, 1H), 5.89 (m, 2H), 4.51 (s, 1H), 4.46 (s, 1H), 4.22 (m, 1H), 3.78, 3.76 (s, s, 3H), 3.22 (m, 2H), 2.28-2.14 (m, 2H), 1.51 (s, 9H); ¹⁹F decH δ −67.96, −68.96; [a]²²D −24.02 (c 0.85, MeOH); ES MS calc. for C₃₁H₃₃FN₂O₆ 586.23, found 609.3 (M+Na).

The intermediate prepared above (17 mg, 0.029 mmol) was dissolved in a dioxane/HCl solution (4 N, 0.2 mL) and MeOH (1 mL) and stirred at room temperature for 3 hours. The reaction mixture was concentrated and the residue free-based with a concentrated aqueous NaHCO₃ solution. This material was purified by chromatography on silica eluting with 5% MeOH in CH₂Cl₂ to give the product 13 (13 mg, 92%). ¹H NMR (CDCl₃, 600 MHz) δ 7.19-7.15 (m, 1H), 7.10 (m, 1H), 6.93-6.80 (m, 4H), 6.72-6.66 (m, 3H), 6.61 (d, J=8.4 Hz, 1H), 6.58 (d, J=8.3 Hz, 1H), 5.89 (dd, J=4.9, 12.9 Hz, 2H), 4.46 (s, 1H), 4.39 (s, 1H), 3.78, 3.76 (s, s, 3H), 3.20 (m, 2H), 2.29-2.10 (m, 2H); ¹⁹F decH δ −67.82, −68.94; [a]²²D −29.16 (c 0.55, MeOH); ES MS calc. for C₂₆H₂₅N₂O₄ 486.18, found 509.3 (M+Na).

N-(3-(benzo[d][1,3]dioxol-5-yl)-3-(2-methoxyphenyl)propyl)-N-(4-((2-(3-(but-3-yn-1-yl)-3H-diazirin-3-yl)ethyl)amino)benzyl)-2,2,2-trifluoroacetamide 16. Compound 16 can be synthesized using the general procedure by treatment of amine 13 (13 mg, 0.0267 mmol) and aldehyde 15 (5 mg, 0.04 mmol). ¹H NMR (CDCl₃, 600 MHz) δ 7.19-7.15 (m, 1H), 7.10 (m, 1H), 6.93-6.80 (m, 4H), 6.72-6.66 (m, 3H), 6.51 (d, J=8.5 Hz, 1H), 6.47 (d, J=8.5 Hz, 1H), 5.89 (m, 2H), 4.46 (s, 1H), 4.22 (s, 1H), 3.78, 3.76 (s, s, 3H), 3.20 (m, 2H), 2.92 (m, 2H), 2.25-2.04 (m, 2H), 2.01 (m, 3H), 1.80 (m, 2H), 1.67 (m, 2H); ¹⁹F decH δ −67.79, −68.94; ES MS calc. for C₃₃H₃₃F₃N₂₄O₄ 606.25, found 607.3 (M+H).

### Exemplary Synthesis of Certain Compounds of the Present Technology

### Synthetic Protocol and Characterization Data for AGX-502

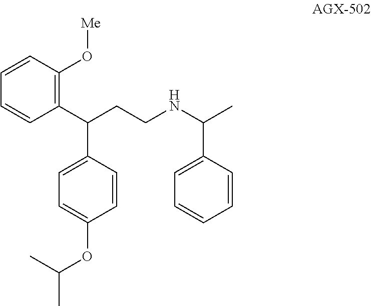 [SMILES: COc1ccccc1C(CCNC(C)c1ccccc1)c1ccc(OC(C)C)cc1]

In accordance with General procedure C (Reductive amination) and similar to the reductive amination depicted in Scheme 2, to a solution of aldehyde 7 (50 mg, 0.168 mmol; see Scheme 2 herein) and 1-phenylethan-1-amine (22 mg, 0.185 mmol) in dichloromethane (1 mL) was added anhydrous magnesium sulfate (30 mg, 0.252 mmol). After stirring for 1 h at reflux, the reaction mixture was filtered and solids washed to remove the drying agent and the filtrate was evaporated in vacuo. The crude imine was then dissolved in methanol (1 mL) and sodium borohydride (13 mg, 0.336 mmol) was added while stirring at 0° C. After additional stirring for 1 h at reflux, the reaction mixture was quenched with water and concentrated to remove methanol. The residue was diluted with dichloromethane, and washed with water. The aqueous layer was extracted with dichloromethane (3 times), and the combined organic layers were washed with saturated aqueous NaCl solution, dried (Na₂SO₄) and concentrated. The residue was purified by ISCO Combi Flash, SiO₂ (4 g) column using 30-50% ethyl acetate/hexanes) gradient to give the product AGX-502 (54 mg, 68% yield.). ¹H NMR (CDCl₃, 500 MHz) δ 7.29-7.17 (m, 5H), 7.16-7.09 (m, 4H), 6.87 (t, J=7.5 Hz, 1H), 6.80 (dd, J=4.0, 8.1 Hz, 1H), 6.73 (m, 2H), 4.47 (m, 1H), 4.37 (m, 1H), 3.74 (d, J=4.3 Hz, 3H), 3.67 (m, 1H), 2.46 (m, 2H), 2.12 (m, 2H), 1.28 (m, 6H); ES MS calc. for C₂₇H₃₃NO₂ 403.25, found 404.23 (M+H).

### Synthetic Protocol and Characterization Data for the Hydrochloride Salt of AGX-502 (“AGX-502·HCl”)

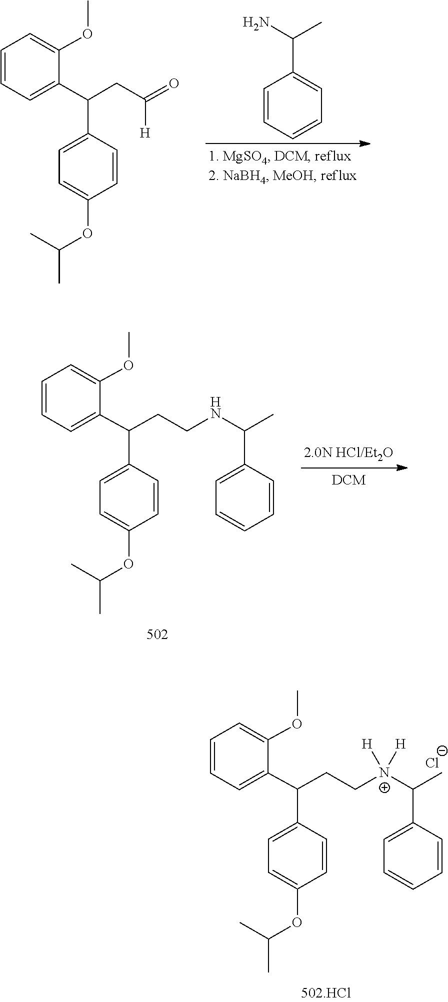

To a solution of the aldehyde 7 (3.0 g, 10.07 mmol; see Scheme 2 herein) and primary amine 1-phenylethyl-1-amine (1.342 g, 11.07 mmol, 1.1 eq) in dichloromethane (65 mL) was added powdered anhydrous magnesium sulfate (1.817 g, 15.11 mmol, 1.5 eq). A water condenser was applied to the round bottom flask and the suspension was vigorously stirred and heated up to reflux (about 48° C.) in an oil bath for 1 hour. The reaction mixture was then cooled down to ambient temperature and filtered through celite and the solid was washed with dichloromethane. The filtrate was evaporated under reduced pressure. The residual crude imine was then dissolved in anhydrous methanol (45 mL) under an argon atmosphere and sodium borohydride (0.762 g, 20.14 mmol, 2.0 eq) was added while stirring at 0° C. The cold bath was then removed, and temperature was brought up to reflux. After stirring for 1 hour at reflux (78° C., oil bath), the reaction mixture was cooled down to room temperature and quenched with water then methanol was removed under vacuum. The residue was diluted with dichloromethane and washed with water. The aqueous layer was extracted with dichloromethane (3×). The combined organic layers were further washed with saturated NaCl, dried over Na₂SO₄ and concentrated under reduced pressure. The residue was purified by flash chromatography, ISCO Combi Flash, SiO₂ (60 g) column using the gradient 0-10% MeOH/DCM (v/v) to give the free amine (AGX-502; 4 g) with >98% yield as a light-yellow thick oil. ¹H NMR (CDCl₃, 600 MHz) δ 7.29-7.18 (m, 5H), 7.16-7.10 (m, 4H), 6.87 (dt, J=2.9, 7.5 Hz, 1H), 6.81 (dd, J=4.7, 8.0 Hz, 1H), 6.74 (dd, J=2.3, 8.6 Hz, 2H), 4.47 (m, 1H), 4.36 (m, 1H), 3.75, 3.74 (s, s, 3H, OMe), 3.67 (m, 1H), 2.43 (m, 2H), 2.18-2.07 (m, 2H), 1.29 (m, 9H); ES MS calc. for C₂₇H₃₃NO₂ 403.25, found 404.15 (M+H). To the free amine AGX-502 (4 g, 9.9 mmol) in solution in dichloromethane (17 mL), 2.0 N HCl in ether (5.98 mL, 11.97 mmol, 1.1 eq) was added dropwise at 0° C. The mixture was stirred for 2 h at room temperature, then the volatiles were removed under vacuum to leave a sticky oil. It was then treated with hexane twice and concentrated in vacuo. The resulting foaming solid was treated with a mixture of ethyl acetate:hexanes (1:1, v:v) to form a white precipitate. The white solid was filtered over Buchner and washed with hexane to afford the desired AGX-502·HCl salt (4.1 g) as a white powder. ¹H NMR (CDCl₃, 600 MHz) δ 10.20 (br, 1H), 9.81 (br, 1H), 7.52 (m, 2H), 7.39-7.34 (m, 3H), 7.10 (m, 1H), 7.08-7.04 (m, 3H), 6.82-6.77 (m, 1H), 6.73 (m, 1H), 6.68 (t, J=8.2 Hz, 2H), 4.44-4.38 (m, 1H), 4.21 (m, 1H), 4.11 (m, 1H), 3.71 (s, 1.5H, OMe), 3.68 (s, 1.5H, OMe), 2.67 (m, 1H), 2.58 (m, 3H), 1.71 (dd, J=6.3, 6.7 Hz, 3H), 1.27 (d, J=6.1 Hz, 3H), 1.23 (t, J=5.9 Hz, 3H); ES MS calc. for C₂₇H₃₃NO₂ 403.25, found 404.22 (M+H); high res MS calc. for C₂₇H₃₄NO₂ 404.2590, found 404.2591 (M+H).

### Synthetic Protocol and Characterization Data for AGX-503

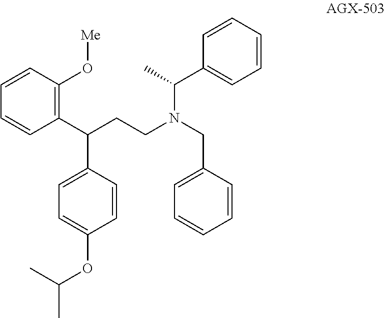 [SMILES: COc1ccccc1C(CCN(Cc1ccccc1)[C@H](C)c1ccccc1)c1ccc(OC(C)C)cc1]

In accordance with General procedure C (Reductive amination) and similar to the reductive amination depicted in Scheme 2, a mixture of aldehyde 7 (50 mg, 0.167 mmol) and (R)—N-benzyl-1-phenylethan-1-amine (32 mg, 0.152 mmol) in acetic acid (10 μL) and ClCH₂CH₂Cl (2 ml) was stirred at rt for 30 min before adding sodium triacetoxyborohydride (75 mg, 0.353 mmol). The resulting mixture was stirred at rt for 18 h. After concentration in vacuo, the mixture was purified flash chromatography, silica gel column to give AGX-503. ¹H NMR (CDCl₃, 500 MHz) δ 7.35-7.22 (m, 8H), 7.19 (m, 2H), 7.09 (t, J=7.8 Hz, 1H), 7.04-6.98 (m, 3H), 6.83-6.75 (m, 2H), 6.68 (dd, J=3.1, 8.7 Hz, 2H), 4.44 (m, 1H), 4.30 (m, 1H), 3.87 (m, 1H), 3.71 (d, d, J=7.1 Hz, 3H), 3.60 (d, J=14 Hz, ½H), 3.50 (s, 1H), 3.43 (d, J=14 Hz, ½H), 2.50 (m, 2H), 2.33 (m, 2H), 2.08-2.04 (m, 2H), 1.27 (m, 9H); ES MS calc. for C₃₄H₃₉NO₂493.30, found 494.23 (M+H).

### Synthetic Protocol and Characterization Data for AGX-504

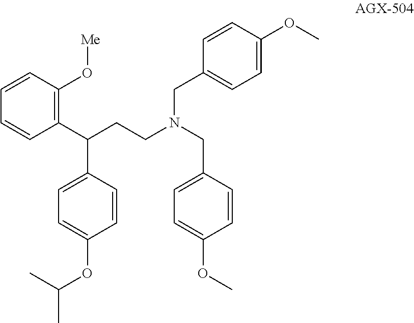 [SMILES: COc1ccc(CN(CCC(c2ccc(OC(C)C)cc2)c2ccccc2OC)Cc2ccc(OC)cc2)cc1]

AGX-504 was synthesized similar to AGX-503 and isolated as the trifluoroacetic acid salt after HPLC purification. AGX-504: ¹H NMR (CDCl₃, 500 MHz) δ 7.26-7.15 (m, 5H), 7.00 (m, 3H), 6.68-6.73 (m, 8H), 4.48 (m, 1H), 4.33 (m, 2H), 4.15 (m, 1H), 3.99 (m, 2H), 3.82 (s, 6H), 3.75 (s, 3H), 2.80 (m, 2H), 2.42 (m, 2H), 1.31 (d, J=6 Hz, 6H); ES MS calc. for C₃₅H₄₁NO₄ 539.30, found 540.23 (M+H).

### Synthetic Protocol and Characterization Data for AGX-505

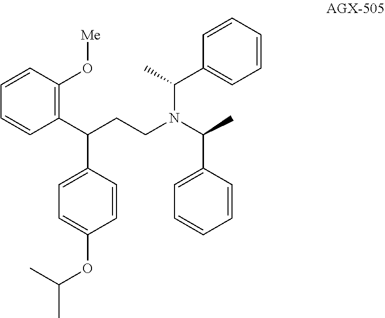 [SMILES: COc1ccccc1C(CCN([C@H](C)c1ccccc1)[C@@H](C)c1ccccc1)c1ccc(OC(C)C)cc1]

AGX-505 (a mixture of diastereomers) was synthesized similar to AGX-503 except using (S)-1-phenyl-N—((R)-1-phenylethyl)ethan-1-amine. AGX-505: ¹H NMR (CDCl₃, 600 MHz) δ 7.28 (m, 8H), 7.20 (m, 2H), 7.08 (m, 1H), 6.90-6.85 (m, 3H), 6.84-6.74 (m, 2H), 6.67 (dd, J=1, 9, 6.8 Hz, 2H), 4.45 (m, 1H), 4.09 (t, J=7.2 Hz, 1H), 3.92 (q, 6.8 Hz, 2H), 3.71 (s), 3.68 (s, together with 3.71 ppm 3H), 2.54 (m, 1H), 2.34 (m, 1H), 1.93-1.78 (m, 2H), 1.29 (m, 12H); ES MS calc. for C₃₅H₄₁NO₂ 507.31, found 508.23 (M+H).

### Synthetic Protocol and Characterization Data for AGX-506

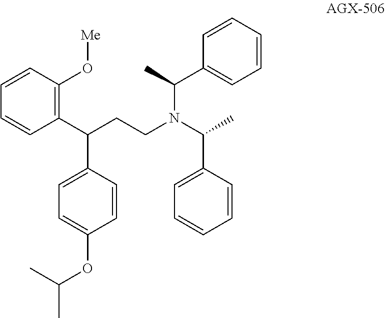 [SMILES: COc1ccccc1C(CCN([C@H](C)c1ccccc1)[C@@H](C)c1ccccc1)c1ccc(OC(C)C)cc1]

AGX-506 (a mixture of diastereomers) was synthesized similar to AGX-505. AGX-506: ¹H NMR (CDCl₃, 600 MHz) δ 7.58 (m, 2H), 7.42-7.36 (m, 6H), 7.21-7.13 (m, 3H), 6.91-6.77 (m, 5H), 6.72 (dd, J=6.7, 8.6 Hz, 2H), 4.49-4.41 (m, 3H), 3.98 (m, 1H), 3.74-3.72 (s, 3H), 3.18 (m, 1H), 2.94 (m, ½H), 2.83 (m, ½H), 2.29-2.20 (m, 1H), 2.00-1.89 (m, 1H), 1.71 (m, 6H), 1.30 (d, J=6.0 Hz, 6H); ES MS calc. for C₃₅H₄₁NO₂ 507.31, found 508.23 (M+H).

### Synthetic Protocol and Characterization Data for AGX-507

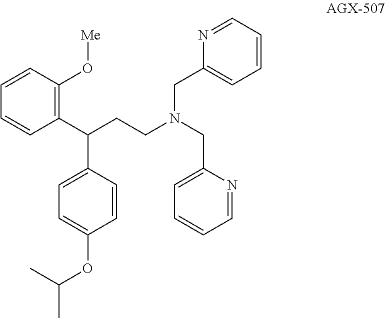 [SMILES: COc1ccccc1C(CCN(Cc1ccccn1)Cc1ccccn1)c1ccc(OC(C)C)cc1]

AGX-507 was synthesized similar to AGX-503 and isolated as the trifluoroacetic acid salt after HPLC purification. AGX-507: ¹H NMR (CDCl₃, 500 MHz) δ 8.73 (d, J=4.7 Hz, 2H), 7.97 (dt, J=1.8, 7.8 Hz, 2H), 7.73 (d, J=7.9 Hz, 2H), 7.50 (t, J=5.8 Hz, 2H), 7.15-7.06 (4H), 6.65 (t, J=7.4 Hz, 1H), 6.79 (d, J=8.2 Hz, 1H), 6.73 (d, J=8.6 Hz, 2H), 4.49-4.37 (m, 5H), 4.27 (t, J=7.8 Hz, 1H), 3.73 (s, 3H), 2.89 (t, J=7.7 Hz, 2H), 2.40 (m, 2H), 1.31 (d, J=6.0 Hz, 6H); ES MS calc. for C₃₁H₃₅N₃O₂ 481.27, found 482.23 (M+H).

### Synthetic Protocol and Characterization Data for AGX-508

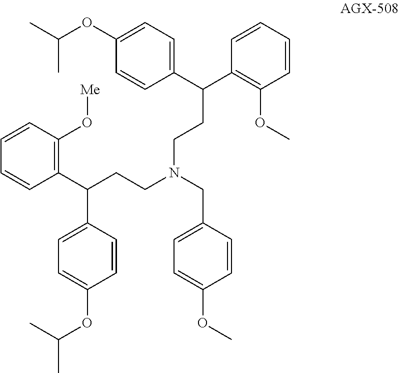 [SMILES: COc1ccc(CN(CCC(c2ccc(OC(C)C)cc2)c2ccccc2OC)CCC(c2ccc(OC(C)C)cc2)c2ccccc2OC)cc1]

AGX-508 was synthesized similar to AGX-503. AGX-508: ¹H NMR (CDCl₃, 500 MHz) δ 7.17-7.04 (m, 10H), 6.85 (t, J=7.4 Hz, 2H), 6.79-6.72 (m, 8H), 4.46 (m, 2H), 4.30 (m, 2H), 3.79 (s, 3H), 3.71 (s, 6H), 3.49 (m, 1H), 2.27 (m, 3H), 2.02 (m, 3H), 1.30 (d, J=6.0 Hz, 12H); ES MS calc. for C₄₆H₅₅NO₅ 701.41, found 702.23 (M+H).

### Synthetic Protocol and Characterization Data for AGX-515

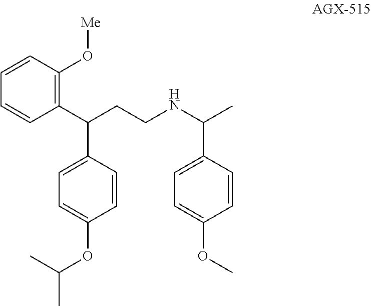 [SMILES: COc1ccc(C(C)NCCC(c2ccc(OC(C)C)cc2)c2ccccc2OC)cc1]

In accordance with General procedure C (Reductive amination) and similar to the reductive amination depicted in Scheme 2, to a solution of aldehyde 7 (1.01 g, 3.356 mmol) and 1-(4-methoxyphenyl)ethan-1-amine (1.1 eq, 0.447 g, 3.69 mmol) in dichloromethane (22 mL) was added anhydrous magnesium sulfate (1.5 eq, 605 mg, 5.034 mmol). After stirring for 1 h at reflux, the reaction mixture was filtered to remove the solid through a pad of celite and the solvent was removed in vacuo. The crude imine was then dissolved in methanol (15 mL) and sodium borohydride (2 eq, 254 mg, 6.712 mmol) was added while stirring at 0° C. After additional stirring for 1 h at reflux, the reaction mixture was quenched with water and concentrated to remove methanol. The residue was diluted with dichloromethane and washed with water. The aqueous layer was extracted with dichloromethane (3 times), the combined organic layers were washed with saturated NaCl, dried over Na₂SO₄ and concentrated. The residue was purified by ISCO Combi Flash, SiO₂ column, using 0-10% MeOH in DCM to give AGX-515, 1.268 g, and 94% yield. ¹H NMR (CDCl₃, 600 MHz) δ 7.16-7.10 (m, 6H), 6.87 (t, J=7.4 Hz, 1H), 6.81 (m, 3H), 6.74 (m, 2H), 4.45 (m, 1H), 4.36 (m, 1H), 3.78-3.74 (s, s, s, 6H), 3.62 (m, 1H), 2.24 (m, 2H), 2.18-2.06 (m, 2H), 1.32-1.25 (m, 9H); ES MS calc. for C₂₈H₃₅NO₃ 433.26, found 434.31 (M+H).

### Exemplarily Biological Experimental Models and Subject Details

In vivo animal studies. Animal studies will be carried out in accordance with institutional regulations (MOI6M130 for the CNV neovascularization studies and M016M138 for the ROP studies) in a non-blinded fashion.

Pharmacokinetic analyses. To determine the pharmacokinetic parameters of compounds of the present technology, eight-week old male Balb/c mice (Taconic farms) will be dosed once by i.p. injection with 30 mg/kg, 50 mg/kg or 100 mg/kg of a compound of the present technology in 70% DMSO (n=3 mice per group). Another set of three mice will be dosed with 100 mg/kg a compound of the present technology prepared in 100% DMSO. Blood will be collected at 30 minutes, 1 hour, 3 hours, 6 hours and 24 hours post administration and plasma analyzed via LC-MS. Following blood collection, the mice will be sacrificed via CO₂ asphyxiation and the eyes from the 30 mg/kg treatment group will be collected and flash frozen for analysis. Data obtained from LC-MS was analyzed via WinNonLin software (version 8.1) for pharmacokinetic parameters.

Toxicity analyses. To assess for toxicity, female athymic nude mice (Envigo), age 6-8 weeks, will be dosed i.p. with either a control vehicle (70% DMSO in water) or a compound of the present technology at 60 mg/kg twice daily for 14 consecutive days. Mice will be sacrificed 24 hours after the last test administration. Gross and complete necropsy, along with clinical pathology analysis will be conducted on all mice. Clinical Chemistry parameters measured may include one or more of: BUN, Creatine, ALP, ALT, AST, GGT, Bilirubin, Total Protein, Albumin, Globulin, Phosphorus, Glucose, Cholesterol, Phosphorus, Calcium, Sodium, Potassium, Chloride. Hematology parameters measured may include one or more of: white blood cells (lymphocytes, Monocytes, Eosinophils, Basophils, Neutrophils), red blood cells, Hemoglobin, Hematocrit, MCV, MCH, MCHC, RDW, Platelets. Organs/Tissues that may be analyzed include one or more of: lung, heart, thymus, kidneys, liver, spleen, gall bladder, pancreas, duodenum, jejunum, ileum, cecum, colon, bone marrow, femur, tibia, sternum, brain, eyes, ears, nasal and oral cavities, teeth, mesenteric and tracheal lymph nodes.

Mouse models of ocular neovascularization. CNV will be induced as previously described. Tobe et al., Am. J. Pathol., 153, 1641-1646 (1998). Briefly, 4-6 week-old female Id1^{-/-} or Id3^{-/-} mice and littermate control Id1^{-/- or Id}3^{-/- mice (all in C}57BL6 background) will have laser-induced rupture of Bruch's membrane at three locations in each eye and after 14 days mice will be euthanized, eyes will be removed, and choroidal flat mounts will be stained with FITC-labeled Griffonia simplicifolia lectin (Vector Laboratories, Burlingame, CA) which selectively stains vascular cells (n=12 mice per group). Flat mounts will be examined by fluorescence microscopy and the area of each CNV was measured by image analysis with Image-Pro Plus software (Media Cybernetics, Silver Spring, MD) by an observer masked with respect to experimental groups. In other experiments, wild type 4-6 week-old, female, C57BL6 mice will have rupture of Bruch's membrane in each eye followed by intravitreal injection of 1-30 μg of a compound of the present technology or vehicle in one eye immediately and after 7 days or mice will be given twice daily i.p. injections of 500 μg of the compound of the present technology or vehicle for 14 days (n=7-10 mice per group). The area of CNV will be measured 14 days after rupture of Bruch's membrane. For Aflibercept+ compound of the present technology treatment experiments, rupture of Bruch's membrane will be carried out and then mice will be treated with 40 μg of Aflibercept, 1-10 μg of a compound of the present technology, a combination of Aflibercept and compound of the present technology, or DMSO. The mice will be 4-6 week-old female C57BL/6. After 14 days, the mice will be euthanized and CNV was measured as described above.

In the ROP experiment 26 C57BL/6 pups, 36 Id^{-/-} and 22 Id3^{-/-} pups will be placed in 75% O₂ at P7. On P12 the mice will be returned to room air and at P17 they will be euthanized and retinal neovascularization will be measured as described above. In the ROP experiment with a compound of the present technology, C57BL6 pups will be placed in 75% 02 at P7. On P12 the mice will be returned to room air and injected in the eye with 1-10 μg of a compound of the present technology or DMSO in the FE (N=15 mice/group). On P17 they will be euthanized and retinal neovascularization will be measured as described above.

Cell lines. HCT116 (male), 4T1 (female) and 293T (female) cell lines will be purchased from ATCC (Manassas, VA, USA) and grown in RPMI (HCT116) or DME (4T1 and 293T) media supplemented with 10% FBS (fetal bovine serum), 1% penicillin-streptomycin and 1% L-Glutamine. HUVECs will be purchased from Corning (sex not specified) (Oneonta, NY, USA) and grown in EGM-2 media (Lonza, Walkersville, MD, USA). Cells will be cultured at 37° C.

Bacterial strains. Id1 and Id3 will be purified from Rosetta 2 (DE3) Competent Cell (Sigma Millipore).

Cell viability assays. Cell lines will be seeded in a 96 well plate (5000 cells per well). After overnight incubation, cells will be treated with a compound of the present technology and incubated for 24 hours then MTT reagent (5 mg/mL) will be added per well and the cells will be incubated for four hours. Following incubation, media will be aspirated and 200 μL DMSO will be added per well. Absorbance will then be measured at 570 nm using a plate reader (Synergy 2, BioTek). Cell growth profiles will be determined by seeding 38,000 cells in a 24-well plate, in triplicate for each time point, and counting the cells on days 1, 3 and 5 after seeding, using trypan blue exclusion of dead cells.

Cell cycle analysis. Cells will be treated with a compound of the present technology or DMSO, collected by trypsinization, washed with 1×PBS, resuspended in 500 μL 1×PBS and then diluted with 6 mL 70% ethanol and stored at −20° C. until analysis. For cell cycle analysis cells will be centrifuged 1000 rpm for 5 minutes, washed with 1×PBS and then resuspended in 0.5 mL PI/RNase staining buffer (550825, BD Biosciences), incubated for 15 minutes at room temperature and analyzed by flow cytometry (LSR II).

HUVEC cell branching assays. For branching assays, 350 μL of Matrigel will be loaded into each well of a 24-well plate on ice and then will incubate the plate for 30 minutes at 37° C. to allow the Matrigel to solidify. 80,000 HUVECs in 0.5 mL of EGM-2 medium with a predetermined concentration of a compound of the present technology will be plated on the solidified Matrigel. At 18-20 hours of incubation when the tube formation is expected to peak, media from the well will be carefully removed and fixed with 10% buffered formalin for 15 minutes. Each well will be washed with DPBS. The morphology of capillary like structures will be visualized using an inverted microscope and will be photographed with a digital camera at 10× magnification. To quantify the tube network, Image J with the Angiogenesis Analyzer plugin (public domain Java-based image-processing program, reference: Carpentier G. ImageJ contribution: Angiogenesis Analyzer. ImageJ News. 2012) will be used and the analysis for the number of nodes, junctions, meshes and total branching length will be performed as per the instruction. The statistical data analyses will be performed using the Wilcoxon test.

HUVEC scratch assay. HUVECs will be seeded on 24-well plate coated with 0.1% fibronectin. After 24 hours when cells were grown to confluency, cells will be serum starved for 4 hours in Endothelial Basal Medium (EBM, Lonza) and then will be scraped with a sterile P200 pipette tip to generate a cell free zone. Cells will be washed with PBS and stimulated with EGM-2 medium with a predetermined concentration of a compound of the present technology for 24 hours. The scratched area at 0 and at 24 hours will be visualized using an inverted microscope and will be photographed with a digital camera at 20× magnification.

Quantification and statistical analysis. Three replicates will generally be used for each experimental condition for in vitro experiments and 5 mice per group will typically be used in each mouse experiment. The sample sizes will be determined based on an expected large effect size. With 3 replicated per condition, an effect size as small as 3 can be detected with 80% power at a two-sided significance level of 0.05 using a two-sample t-test. With 5 mice per group, an effect size as small as 2 can be detected with 80% power at a two-sided significance level of 0.05 using a two-sample t-test. Additional experiments may be performed when larger variation in data is observed and data will be pooled for analysis. In general, Welch's t-test will be used to examine differences between two groups. ANOVA will be used to examine differences across multiple experimental groups. Data may be transformed to ensure the underlying normality assumptions were met. Weighted linear regression analysis will be used when heteroscedasticity is observed and data points in each group will typically be weighted by the reciprocal of the standard deviation of data in each group. For data pooled from multiple experiments, the model will include both experiments and experiments by treatment group interaction as covariates to account for potential differences in experiments. Significance of linear contrasts of interest will be assessed based on estimates obtained from the weighted least squares. Q-Q plot of the residuals will be examined to ensure the underlying model assumptions were met. P-value <0.05 will be considered statistically significant.

### Example 1. Exemplary Biological Data for AGX-502

In accordance with the protocols described above, AGX-502 exhibited significant activity against ID1 in cells and was shown to be very active both by Western Blot and cell killing. In fact, AGX-502 exhibited complete cell killing by 8 hours, significantly faster than AGX-A which showed complete cell killing by 24 hours. Initial testing shows a significantly favorable IC50 (24 h) for AGX-502.

### Example 2. Effects of AGX51. AGX-A. and Compounds of the Present Technology on Various Additional Cancer Cell Lines

The 4T1 murine mammary tumor cell line and nine breast cancer cell lines representing the major breast cancer subtypes (ER+, HER2+, and TNBC) including MDA-MB-157, MDA-MB-436, MDA-MB-231, MDA-MB-453, MDA-MB-361, BT-474, SK-BR-3, MCF-7 and T47-D) will be grown in RPMI or DMEM (Dulbecco's Modified Eagle Medium) media supplemented with 10% FBS (fetal bovine serum), 1% penicillin-streptomycin and 1% L-Glutamine. Patient derived xenograft cell lines BR7, BR11, and IBT will be established directly from breast cancer bone metastases specimens surgically resected from patients with informed consent. BR7 and BR11 specimens will be obtained from ER positive (HER2 negative and PR negative) metastatic breast cancer patients, while the IBT specimen will be obtained from a metastatic, triple negative breast cancer patient. Fresh tumor tissues will be quickly washed with ice cold PBS and minced into about 1-3 mm pieces in MEM medium (without FBS) using sterile razor blades. A fraction of the minced original tumor tissues will be incubated with Collagenase/Hyaluronidase enzyme mix (1,000 Units, Voden Medical, Lombardia, Italy) in MEM medium without FBS (5 mL/250 mg tissue) for 2-4 hours. The dissociated tumor tissues will then be filtered throughout a 70 μm nylon filter, and cells will be concentrated by centrifugation at room temperature and seeded to derive respective primary cell cultures in MEM medium with 3% FBS (Sigma). The primary cell cultures will be transduced with the fluorescent td tomato-/EGFP-luciferase fusion protein expressing lentiviral vectors for 18-24 hours, the primary cells will then be maintained in MEM media supplemented with 3% FBS, 1% penicillin-streptomycin and 1% L-Glutamine. Aliquots of the primary cell cultures will also cryopreserved following a minimal number (3-4) of in vitro passages. The cells will be treated with either 100 μM DMSO, AGX51, AGX-A, or a compound of the present technology. The cell morphology and growth will be monitored daily for 1 week by microscopy for changes in morphology or cell death. For IC50 determination, cancer cell lines will be seeded in a 96 well plate (5000 cells per well). After overnight incubation, cells will be treated with AGX51 (0, 5, 10, 20, 40, 60 μM), AGX-A (0, 5, 10, 20, 40, 60 μM), or with a compound of the present technology (0, 5, 10, 20, 40, 60 μM) and incubated for 24, 48 and 72 hours, each condition being done in triplicate. At each time point 40 μL of MTT reagent (5 mg/mL) will be added per well and the cells will be incubated for four hours. Following incubation, media will be aspirated and 200 μL DMSO added per well. Absorbance will then measured at 570 nm using a plate reader (Synergy 2, BioTek).

The effect of DMSO, AGX51, AGX-A, or a compound of the present technology on 4T1 cells will also be assessed using Alamar blue viability assays according to manufacturer's instructions. Briefly, 5000 4T1 cells will be seeded in a 96-well plate, and the next day will be treated with 40 μM AGX51 for 24 hours, after which point a 1:10 dilution of Alamar blue cell viability reagent will be added to the cells and absorbance will be measured 2, 3, 4, 5 and 6 hours after addition of the reagent using a plate reader (Synergy 2, BioTek).

The effect of DMSO, AGX51, AGX-A, or a compound of the present technology on pancreatic cancer cell and organoid lines will also examined. Lines to be examined are human pancreatic cancer cell line Panc1 and A21, mouse pancreatic cancer lines 806 (KrasG12D; Ink4a−/−; Smad4−/−), NB44 (KrasG12D; Ink4a−/−) and 4279 (KrasG12D; Ink4a−/−), and mouse pancreatic organoid cell lines T7 and T8. Pancreatic spheroids will be grown in Ultra Low Attachment Culture plates (Corning, Oneonta, NY, USA) in DMEM supplemented with Glutamax (2 mM) and heparin (5 μg/mL). Pancreatic organoids will be embedded in Matrigel with Advanced DMEM/F12 (12634-028, Gibco, Carlsbad, Calif.) supplemented with B-27 (12587-010, Life Technologies, Carlsbad, CA), HEPES (10 mM), 50% Wnt/R-spondin/Noggin-conditioned medium (ATCC, CRL-3276), Glutamax (2 mM, Invitrogen, Carlsbad, Calif.), N-acetyl-cysteine (1 mM, Sigma, St. Louis, MO, USA), nicotinamide (10 mM, Sigma, St. Louis, MO, USA), epidermal growth factor (50 ng/mL, Peprotech, Rocky Hill, NJ), gastrin (10 nM, Sigma-Aldrich, St. Louis, MO), fibroblast growth factor-10 (100 ng/mL, Peprotech, Rocky Hill, NJ), A83-01 (0.5 μM, Tocris, Bristol, United Kingdom) as described by Boj, S. F., et al., Cell 160: 324-338 (2015), incorporated herein by reference. All cell lines and organoids will be maintained at 37° C. and 5% CO₂. Cell viability will be measured using Cell Titer-Glo (Promega, Madison, WI) according to manufacturer's instructions. It is expected that compounds of the present technology will exhibit similar or significantly improved effects over AGX51 and/or AGX-A, providing yet further evidence that the compounds of the present technology are useful in treating cancers.

### Example 3. Effects of AGX51. AGX-A. and Compounds of the Present Technology on Cancer Cell Line Xenografts in Mice

Effects of DMSO, AGX51, AGX-A, or a compound of the present technology on primary tumors will be tested with and without paclitaxel in a primary tumor xenograft model using MDA-MB-231 cells. Orthotopic mammary fat pad tumors will be generated by injecting 5×10⁶ MDA-MB-231 cells (in 1:1 PBS:Matrigel) into the right caudal mammary fat pad of 8-12 week-old, female athymic nu/nu mice. Mice will be obtained from Simonsen Laboratories, Gilroy, CA. Tumors will be allowed to grow to ˜100 mm³, at which point they will be divided into 12 groups of 5 mice with approximately the same tumor burden and treatment will then be initiated. Group 1 will be vehicle (DMSO, dosed q5d) control, Group 2 will receive 5 days of 15 mg/kg paclitaxel once a day, Group 3 will receive 19 days of 60 mg/kg AGX51 twice a day, Group 4 will receive 19 days of a combination of 15 mg/kg paclitaxel once a day and 6.7 mg/kg AGX51 twice a day, Group 5 will receive 19 days of a combination of 15 mg/kg paclitaxel once a day and 20 mg/kg AGX51 twice a day, Group 6 will receive 19 days of a combination of 15 mg/kg paclitaxel once a day and 60 mg/kg AGX51 twice a day, Group 7 will receive 15 mg/kg paclitaxel once a day for 19 days and concurrently receive 60 mg/kg AGX51 twice a day for the first 7 days, Group 8 will receive 19 days of 60 mg/kg of a compound of the present technology twice a day, Group 9 will receive 19 days of a combination of 15 mg/kg paclitaxel once a day and 6.7 mg/kg compound of the present technology twice a day, Group 10 will receive 19 days of a combination of 15 mg/kg paclitaxel once a day and 20 mg/kg compound of the present technology twice a day, Group 11 will receive 19 days of a combination of 15 mg/kg paclitaxel once a day and 60 mg/kg compound of the present technology twice a day, Group 12 will receive 15 mg/kg paclitaxel once a day for 19 days and concurrently receive 60 mg/kg compound of the present technology twice a day for the first 7 days. Treatments will be administered i.p. Tumor volumes will be determined throughout the study using a digital caliper and the formula: tumor volume=½(length×width2) where the greatest longitudinal diameter is the length of the tumor and the greatest transverse diameter is the width. At study termination, the mice will be sacrificed by cervical dislocation. It is expected that compounds of the present technology will exhibit similar or significantly improved effects as compared to AGX51 and/or AGX-A and provide yet further evidence that the compounds of the present technology are useful in treating triple negative breast cancer.

Effects of DMSO, AGX51, AGX-A, or a compound of the present technology on metastasis will also be examined using a lung colonization model. Lung metastases will be generated by injecting 6-8 week-old, female, Balb/c mice with 50,000 luciferase-labeled 4T1 cells into the tail vein. Twenty-four hours after tail vein injections, mice will be treated once a day with DMSO, 50 mg/kg AGX51, 50 mg/kg AGX-A, or 50 mg/kg of a compound of the present technology twice a day (at least five mice per treatment group) by i.p. injection. Development of lung metastases will be monitored using the IVIS-200 in vivo imaging system. Effects of DMSO, AGX51, AGX-A, or a compound of the present technology on established lung metastases will also be examined. Once evidence of lung metastases is observable by in vivo imaging, mice will be divided into groups of five mice each with approximately the same tumor burden per treatment group. The groups will be: Group 1 vehicle (DMSO) for 5 days, Group 2 50 mg/kg AGX51 twice a day for 5 days, Group 3 15 mg/kg paclitaxel once a day for 5 days, Group 4 a 5 day combination of 50 mg/kg AGX51 twice a day and 15 mg/kg paclitaxel once a day, Group 5 50 mg/kg AGX-A twice a day for 5 days, Group 6 a 5 day combination of 50 mg/kg AGX-A twice a day and 15 mg/kg paclitaxel once a day, Group 7 50 mg/kg compound of the present technology twice a day for 5 days, and Group 8 a 5 day combination of 50 mg/kg compound of the present technology twice a day and 15 mg/kg paclitaxel once a day. At the end of the experiments mice will be euthanized and tissues were collected for further analyses. Lung tumor burden will be quantified in a blinded fashion by a pathologist. It is expected that compounds of the present technology will exhibit similar or significantly improved effects as compared to AGX51 and/or AGX-A.

To assess the inhibition of the extravasation and initial seeding at the secondary site or the progression of extravasated cancer cell outgrowth into tumors, mice will be injected with GFP-labeled 4T1 cells from the tail vein, and treated with DMSO, AGX51 (50 mg/kg), AGX-A (50 mg/kg), or a compound of the present technology (50 mg/kg) for 24 or 48 hours, then the lungs will be stained for GFP (i.e., tumor cells) and tumor cell number will be quantified. It is expected that compounds of the present technology will exhibit similar or significantly improved effects as compared to AGX51 and/or AGX-A.

Effects of DMSO, AGX51, AGX-A, or a compound of the present technology on sporadic tumor will be examined using the azoxymethane (AOM) colon tumor model, a chemically induced autochthonous model of adenoma. Spontaneous colon tumors will be induced by treating 30, 4-week old male A/J mice (Jackson Laboratory) with AOM (10 mg/kg; Sigma Aldrich) once a week by i.p. injection for 6 weeks. Mice will be maintained on AIN-93G purified diet (Research Diets) for the duration of the experiment. After a three-week treatment break mice will be treated i.p. for three weeks with DMSO, AGX51 (15 mg/kg), AGX-A (15 mg/kg), or a compound of the present technology (15 mg/kg). Following the last injection, the mice will be euthanized and colon tumors will be formalin fixed to assess tumor burden. Tumor numbers and size will be determined in whole mounts of the tissues following methylene blue staining. It is expected that compounds of the present technology will exhibit similar or significantly improved effects as compared to AGX51 and/or AGX-A.

### Example 4. Effects of AGX51. AGX-A. and Compounds of the Present Technology on Prostate Cancer Cell Lines

Prostate cancer cell lines DU145 and PC3 (in 10% BCS) will be cultured in Ham's F12 (Gibco, Carlsbad, Calif.) medium containing 10% BCS (Hyclone, Logan, Utah) and appropriate antibiotics (pen/strep, fungizaone, and gentamycin (Invitrogen Inc., Carlsbad, Calif.). All cells will be cultured at 37° C. in a fully humidified atmosphere containing 5% CO₂.

At 50% confluence, the cells will be treated with either 100 μM DMSO, AGX51, AGX-A, or a compound of the present technology. The cell morphology and growth will be monitored daily for 1 week by microscopy for changes in morphology or cell death. Apoptosis will be determined by measuring caspase 3 and caspase 7 activities using the Caspase-Glo 3/7 Assay system from Promega (Madison, Wis.).

It is expected that AGX51, AGX-A, and compounds of the present technology will show a pronounced effect on DU145 cells: after three days, the cells may appear very unhealthy and will be unable to proliferate as compared to the controls. Additionally, after six days, treatment of DU145 cells with AGX51, AGX-A, and a compound of the present technology will lead to cell death. It is expected that compounds of the present technology will exhibit similar or significantly improved effects as compared to AGX51 and/or AGX-A.

The molecular mechanism underlying the effect of the compounds of the present technology on prostate cancer cells will be assessed by measuring the activity of the primary mediators of apoptosis, caspase 3/7. It is expected that low concentrations of a compound of the present technology will result in a significant increase in caspase 3/7 in both DU145 and PC3 cells, which will be higher than the caspase activity in cells treated with staurosporine (10 μm), a known apoptosis inducing agent.

### Example 5. Effects of AGX51. AGX-A. and Compounds of the Present Technology on Anti-Angiogenic Activity in a Matrigel Assay in Mice

VEGF-165 and FGF-2 treated Matrigel plugs will be implanted on Day 0 into C57BL/6 mice. Mice will be treated with either vehicle, AGX51, AGX-A, or a compound of the present technology. The AGX51, AGX-A, and compound of the present technology will be provided either in the plugs (25 μg/mg) or by daily ip treatment (30 or 100 mg/kg) for 10 days. Plugs will be harvested on Day 10, fixed and paraffin embedded. Three sections (5 μM thickness) of each plug will be stained with an anti-CD31 antibody and counterstained with hematoxylin and eosin stain. CD31-positive microvessels will be counted for one entire cross-section per plug and the average micro-vessel density±SD vessels will be determined. Student's t-test will be used for statistical analysis.

It is anticipated that compared to vehicle control animals, treatments with AGX51, AGX-A, or a compound of the present technology will provide significant protection from new blood vessel formation. A typical picture of slices of the Matrigel plugs is expected to show the presence of complete blood vessels in vehicle alone control. In contrast, the presence of endothelial cells is expected to significantly decrease with the treatment with AGX51, AGX-A, or a compound of the present technology. It is expected that compounds of the present technology will exhibit similar or significantly improved effects as compared to AGX51 and/or AGX-A.

### Example 6. Effects of AGX51. AGX-A. and Compounds of the Present Technology on Metastatic Activity in a LLC Mouse Model

Thirty C57BL/6 mice will be implanted with 7.5×10⁵ LLC cancer cells/animal. Seven days after implantation, 5M/5F per group will be treated daily ip for 25 days with dosing vehicle (DMSO), AGX51, AGX-A, or a compound of the present technology. Fourteen days after implantation, another group of 5M/5F animals will be treated daily ip for 18 days with 50 mg/kg of AGX51, another group of 5M/5F animals will be treated daily ip for 18 days with 50 mg/kg of AGX-A, and another group of 5M/5F animals will be treated daily ip for 18 days with a compound of the present technology. Tumors will be measured three times from Day 7 to 14. On Day 14, the tumors will be excised. The animals will be necropsied for the presence of lung metastases on day 32, 18 days post excision. It is expected that the treatment with AGX51, AGX-A, and a compound of the present technology will profoundly diminish lung metastases. Further, it is expected that compounds of the present technology will exhibit similar or significantly improved effects as compared to AGX51 and/or AGX-A.

### Example 7: In Vivo Efficacy of AGX-502 In a Murine Model of Cholangiocarcinoma

An animal model of cholangiocarcinoma predictive of anti-cholangiocarcinoma drug efficacy in humans, as described in Mattar, M. et al. Establishing and Maintaining an Extensive Library of Patient-Derived Xenograft Models. Front Oncol. 2018 Feb. 19; 8:19 (doi: 10.3389/fonc.2018.00019), evidences the dose-dependent anti-cancer efficacy of AGX-502.

### Study Protocol—Animal Model and Experimental Design:

Mice: NSG females, 6-8 weeks old
PDX: RomeP_PHCH_X_0008a, serially transplanted sc with matrigel
Treatment Groups (at least 5 animals/group): treatments started when tumors reached 100 mm³ (“Day 0” for each animal), dosed via oral gavage
   Group 1. Saline with 10% by weight 2-hydroxypropyl-β-cyclodextrin (“Vehicle”), p.o. twice daily for 5 days per week (“BIDx5”) for 4 weeks (“×4 weeks”)
   Group 2. AGX-A 50 mg/kg in Vehicle, p.o. BIDx5×4 weeks
   Group 3. AGX-A 100 mg/kg in Vehicle, p.o. BIDx5×4 weeks
   Group 4. AGX-502 50 mg/kg in Vehicle, p.o. BIDx5×4 weeks
   Group 5. AGX-502 100 mg/kg in Vehicle, p.o. BIDx5×4 weeks.

Tumor volumes, weights, and clinical signs were monitored a minimum of twice per week for each animal in each group throughout the study. Mice were sacrificed by CO₂ asphyxiation once the tumor volume exceeded 2000 mm³ or body weight loss was higher than 10%. Upon completion of the experiment, tumors were harvested and processed for immunoblotting and/or immunohistochemistry assays.

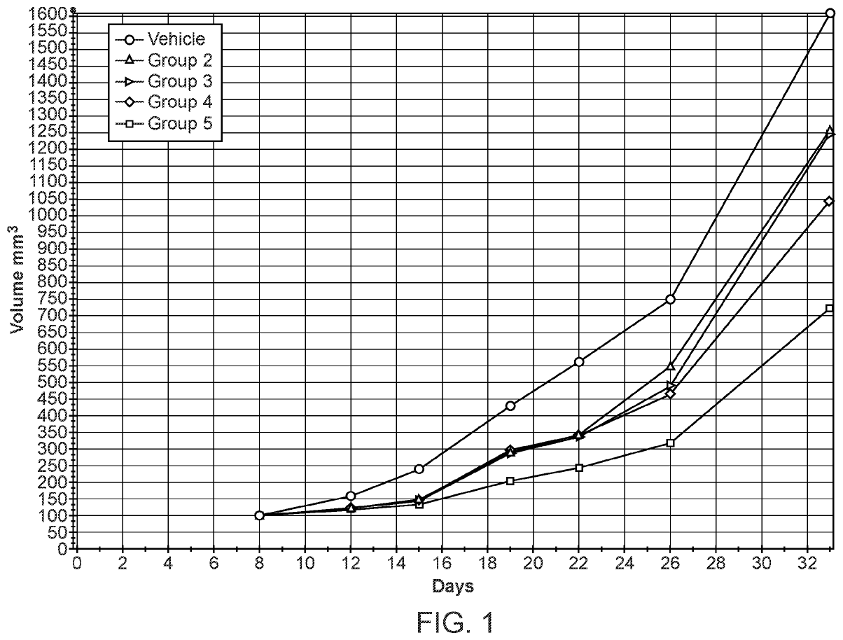

Tumor volume data (averaged for each Group) from this study is presented in FIG. 1. Further, it was observed for each Group that animal weight was essentially unchanged for the duration of the study. These studies evidence that compounds of the present technology are not only well tolerated but further exhibit significant therapeutic properties.

### Example 8: In Vivo Efficacy of Combination Therapy Including Anti-Id Compounds of the Present Technology In a Murine Model of Cholangiocarcinoma

The animal model of cholangiocarcinoma predictive of anti-cholangiocarcinoma drug efficacy in humans discussed in Example 7 is expected to evidence the dose-dependent anti-cancer efficacy of anti-Id compounds of the present technology in combination with chemotherapy drugs. As an example, such a model for AGX-502 and gemcitabine is discussed below.

### Study Protocol—Animal Model and Experimental Design:

Mice: NSG females, 6-8 weeks old
PDX: RomeP_PHCH_X_0008a, serially transplanted sc with matrigel
Treatment Groups (at least 5 animals/group): treatments will be started when tumors reach 100 mm³ (“Day 0” for each animal)
   Group 1. Saline with 10% by weight 2-hydroxypropyl-β-cyclodextrin (“Vehicle”), p.o. twice daily (“BID”)
   Group 2. AGX-502 100 mg/kg in Vehicle, p.o. BID
   Group 3. AGX-502 200 mg/kg in Vehicle, p.o. BID
   Group 4. Gemcitabine 15 mg/kg in Vehicle, i.p. once a week (“QW”)
   Group 5. Gemcitabine 15 mg/kg in Vehicle, i.p. QW+AGX-502 100 mg/kg in Vehicle, p.o. BID

During the study, tumor volumes, weights, and clinical signs will be monitored a minimum of twice per week for each animal in each group. Mice will be sacrificed by CO₂ asphyxiation once the tumor volume exceeds 2000 mm³ or body weight loss is higher than 10%. Upon completion of the study, tumors will be harvested and may be processed for immunoblotting and/or immunohistochemistry assays.

The data from such studies is expected to confirm the significant potency and efficacy of anti-Id compounds of the present technology, both alone as well as part of a combination therapy.

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A compound according to Formula I

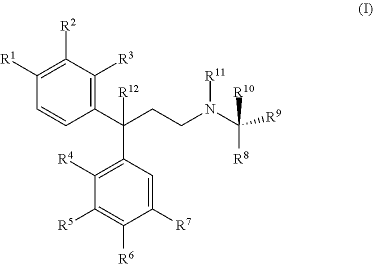

or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹³)(R¹⁴);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹⁵)(R¹⁶)
R⁸ is aryl or heteroaryl;
R⁹ and R¹⁰ are each independently H, C₁-C₃ alkyl, or trifluoromethyl;
R¹¹ is H, C₁-C₃ alkyl, aralkyl, or heteroaralkyl;
at least one of R⁹, R¹⁰, and R¹¹ is not H;
R¹² is H, C₁-C₃ alkyl, or fluoro; and
R¹³, R¹⁴, R¹⁵, and R¹⁶ are each independently C₁-C₃ alkyl.
B. The compound of Paragraph A, wherein R¹, R², and R³ are each independently H, C₁-C₃ alkyl, C₁-C₃ alkoxy, trifluoromethyl, trifluoromethoxy, halo, or —N(R¹⁰)(R¹¹).
C. The compound of Paragraph A or Paragraph B, wherein R¹, R², and R³ are each independently H, C₁-C₃ alkyl, C₁-C₃ alkoxy, halo, or —N(Me)₂.
D. The compound of any one of Paragraphs A-C, wherein R¹, R², and R³ are each independently H, methyl, methoxy, isopropyl, isopropoxy, fluoro, or —N(Me)₂.
E. The compound of any one of Paragraphs A-D, wherein R³ is methoxy.
F. The compound of any one of Paragraphs A-E, wherein R⁴, R⁵, R⁶, and R⁷ are each independently H, C₁-C₃ alkyl, C₁-C₃ alkoxy, trifluoromethyl, trifluoromethoxy, halo, or —N(R²)(R¹³).
G. The compound of any one of Paragraphs A-F, wherein R⁴, R⁵, R⁶, and R⁷ are each independently H, C₁-C₃ alkyl, C₁-C₃ alkoxy, halo, or —N(Me)₂.
H. The compound of any one of Paragraphs A-G, wherein R⁴, R⁵, R⁶, and R⁷ are each independently H, methyl, methoxy, isopropyl, isopropoxy, fluoro, or —N(Me)₂.
I. The compound of any one of Paragraphs A-H, wherein R⁶ is isopropoxy.
J. The compound of any one of Paragraphs A-I, where the compound is of Formula IA

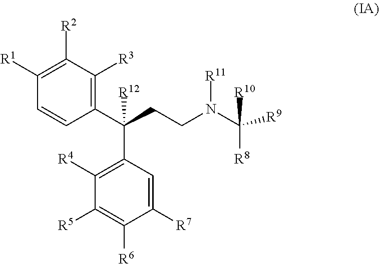

or a pharmaceutically acceptable salt and/or solvate thereof.
K. The compound of any one of Paragraphs A-J, wherein the compound is of Formula IB

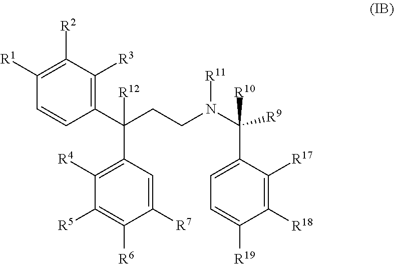

or a pharmaceutically acceptable salt and/or solvate thereof, wherein
R¹⁷, R¹⁸, and R¹⁹ are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido.
L. The compound of Paragraph K, wherein R¹⁷, R¹⁸, and R¹⁹ are each independently H, C₁-C₃ alkyl, C₁-C₃ alkoxy, trifluoromethyl, trifluoromethoxy, halo, aryloxy, aryloyl, or —N(C₁-C₃ alkyl)₂.
M. The compound of Paragraph K or Paragraph L, wherein R¹⁷, R¹⁸, and R¹⁹ are each independently H, C₁-C₃ alkyl, C₁-C₃ alkoxy, trifluoromethyl, trifluoromethoxy, halo, or —N(Me)₂.
N. The compound of any one of Paragraphs K-M, wherein R¹⁷, R¹⁸, and R¹⁹ are each independently H, methyl, methoxy, isopropyl, isopropoxy, fluoro, or —N(Me)₂.
O. The compound of any one of Paragraphs K-N, wherein at least one of R¹⁷, R¹⁸, and R¹⁹ is not H.
P. The compound of any one of Paragraphs K-O, wherein at least one of R¹⁷, R¹⁸, and R¹⁹ is C₁-C₃ alkoxy, trifluoromethyl, trifluoromethoxy, pentafluorosulfanyl, halo, aryloxy, aryloyl, hydroxyl, amino, or amido.
Q. The compound of any one of Paragraphs K-P, wherein R¹⁷, R¹⁸, and R¹⁹ are each independently H.
R. The compound of any one of Paragraphs A-Q, wherein R¹¹ is

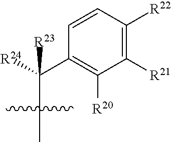

wherein
R²⁰, R²¹, and R²² are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido; and
R²³ and R²⁴ are each independently H, C₁-C₃ alkyl, trifluoromethyl, or aryl, provided at least one of R²³ and R²⁴ is not aryl.
S. The compound of Paragraph R, wherein R²⁰, R²¹, and R²² are each independently H, C₁-C₃ alkyl, C₁-C₃ alkoxy, trifluoromethyl, trifluoromethoxy, halo, aryloxy, aryloyl, or —N(C₁-C₃ alkyl)₂.
T. The compound of Paragraph R and Paragraph S, wherein R²⁰, R²¹, and R²² are each independently H, C₁-C₃ alkyl, C₁-C₃ alkoxy, trifluoromethyl, trifluoromethoxy, halo, or —N(Me)₂.
U. The compound of any one of Paragraphs R-T, wherein R²⁰, R²¹, and R²² are each independently H, methyl, methoxy, isopropyl, isopropoxy, fluoro, or —N(Me)₂.
V. The compound of any one of Paragraphs R-U, wherein at least one of R²⁰, R²¹, and R²² is not H.
W. The compound of any one of Paragraphs R-V, wherein at least one of R²⁰, R²¹, and R²² is C₁-C₃ alkoxy, trifluoromethyl, trifluoromethoxy, pentafluorosulfanyl, halo, aryloxy, aryloyl, hydroxyl, amino, or amido.
X. The compound of any one of Paragraphs R-W, wherein R²⁰, R²¹, and R²² are each independently H.
Y. The compound of any one of Paragraphs A-X, wherein the compound is

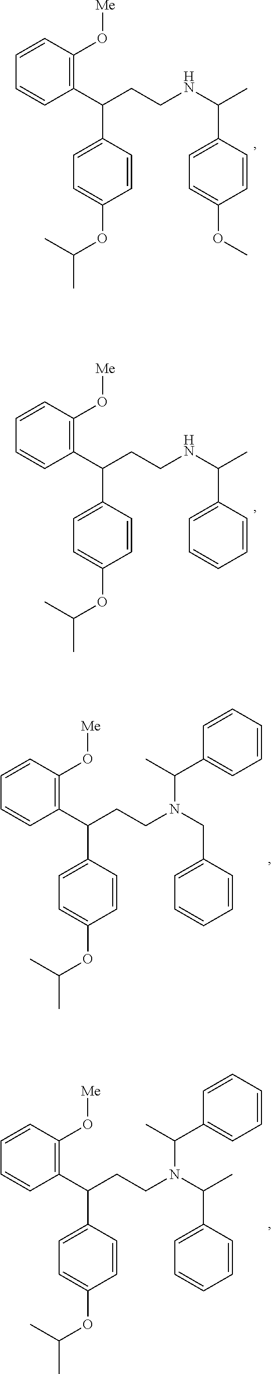 [SMILES: COc1ccc(C(C)NCCC(c2ccc(OC(C)C)cc2)c2ccccc2OC)cc1 ; COc1ccccc1C(CCNC(C)c1ccccc1)c1ccc(OC(C)C)cc1 ; COc1ccccc1C(CCN(Cc1ccccc1)C(C)c1ccccc1)c1ccc(OC(C)C)cc1 ; COc1ccccc1C(CCN(C(C)c1ccccc1)C(C)c1ccccc1)c1ccc(OC(C)C)cc1]

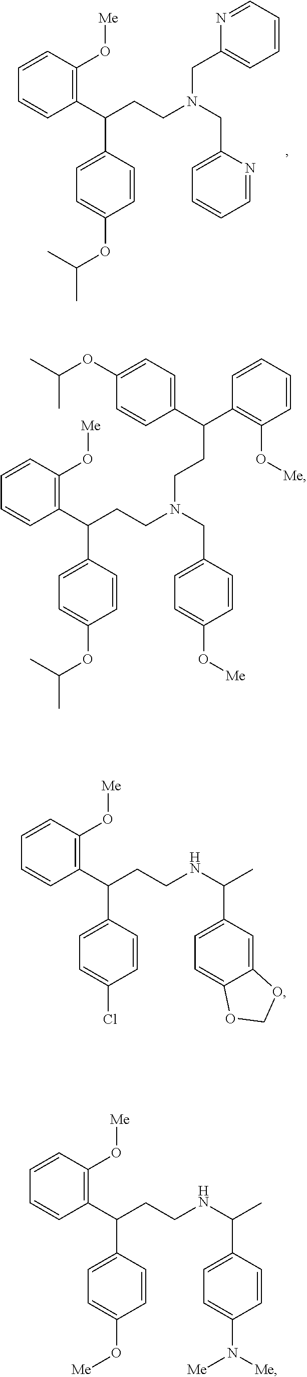 [SMILES: COc1ccccc1C(CCN(Cc1ccccn1)Cc1ccccn1)c1ccc(OC(C)C)cc1 ; COc1ccc(CN(CCC(c2ccc(OC(C)C)cc2)c2ccccc2OC)CCC(c2ccc(OC(C)C)cc2)c2ccccc2OC)cc1 ; COc1ccccc1C(CCNC(C)c1ccc2c(c1)OCO2)c1ccc(Cl)cc1 ; COc1ccc(C(CCNC(C)c2ccc(N(C)C)cc2)c2ccccc2OC)cc1]

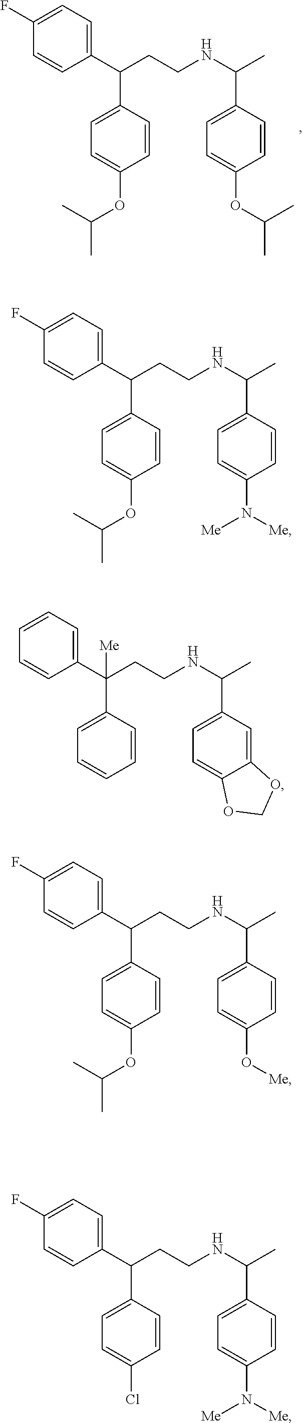 [SMILES: CC(C)Oc1ccc(C(C)NCCC(c2ccc(F)cc2)c2ccc(OC(C)C)cc2)cc1 ; CC(C)Oc1ccc(C(CCNC(C)c2ccc(N(C)C)cc2)c2ccc(F)cc2)cc1 ; CC(NCCC(C)(c1ccccc1)c1ccccc1)c1ccc2c(c1)OCO2 ; COc1ccc(C(C)NCCC(c2ccc(F)cc2)c2ccc(OC(C)C)cc2)cc1 ; CC(NCCC(c1ccc(F)cc1)c1ccc(Cl)cc1)c1ccc(N(C)C)cc1]

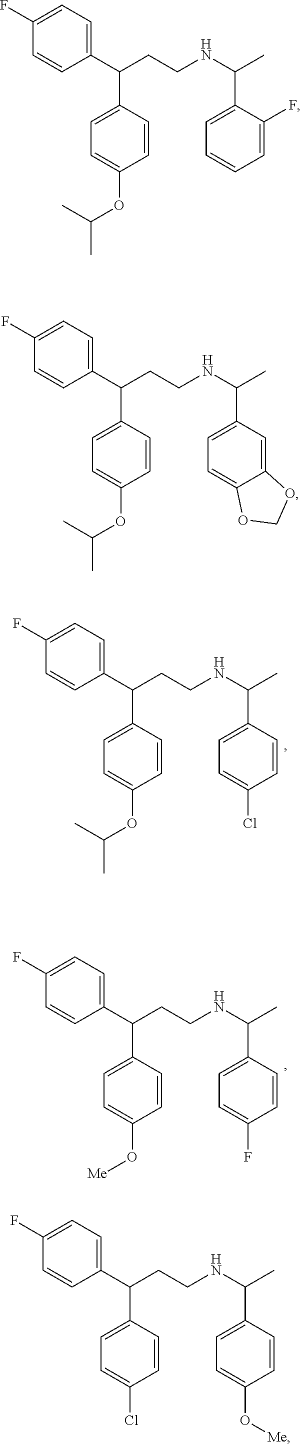 [SMILES: CC(C)Oc1ccc(C(CCNC(C)c2ccccc2F)c2ccc(F)cc2)cc1 ; CC(C)Oc1ccc(C(CCNC(C)c2ccc3c(c2)OCO3)c2ccc(F)cc2)cc1 ; CC(C)Oc1ccc(C(CCNC(C)c2ccc(Cl)cc2)c2ccc(F)cc2)cc1 ; COc1ccc(C(CCNC(C)c2ccc(F)cc2)c2ccc(F)cc2)cc1 ; COc1ccc(C(C)NCCC(c2ccc(F)cc2)c2ccc(Cl)cc2)cc1]

or a pharmaceutically acceptable salt and/or solvate thereof.
Z. The compound of any one of Paragraphs A-Y, wherein the compound is of Formula IC

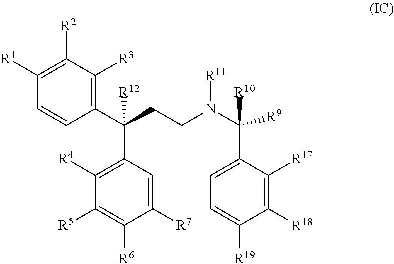

or a pharmaceutically acceptable salt and/or solvate thereof.
AA. The compound of any one of Paragraphs A-Z, wherein the compound is

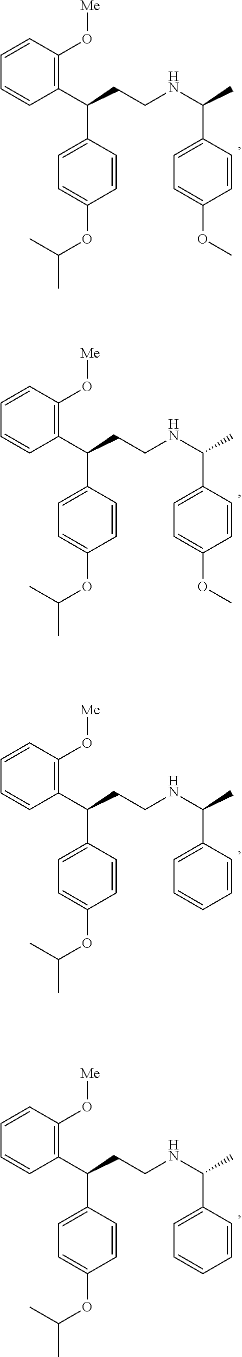 [SMILES: COc1ccc([C@H](C)NCC[C@@H](c2ccc(OC(C)C)cc2)c2ccccc2OC)cc1 ; COc1ccc([C@@H](C)NCC[C@@H](c2ccc(OC(C)C)cc2)c2ccccc2OC)cc1 ; COc1ccccc1[C@@H](CCN[C@@H](C)c1ccccc1)c1ccc(OC(C)C)cc1 ; COc1ccccc1[C@@H](CCN[C@H](C)c1ccccc1)c1ccc(OC(C)C)cc1]

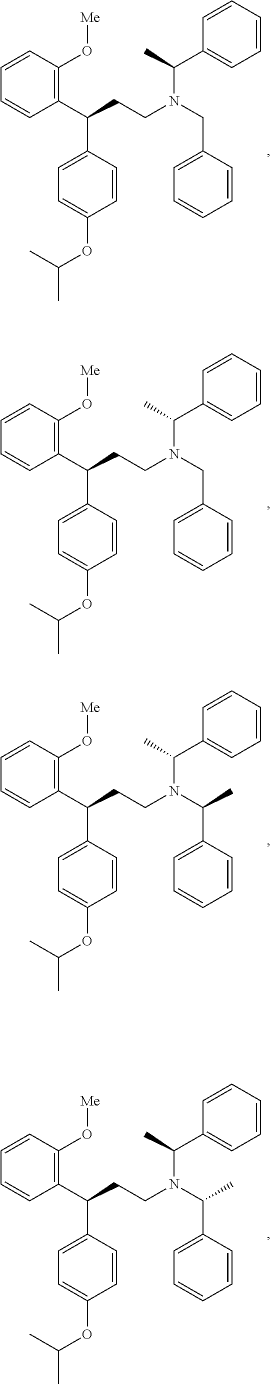 [SMILES: COc1ccccc1[C@@H](CCN(Cc1ccccc1)[C@@H](C)c1ccccc1)c1ccc(OC(C)C)cc1 ; COc1ccccc1[C@@H](CCN(Cc1ccccc1)[C@H](C)c1ccccc1)c1ccc(OC(C)C)cc1 ; COc1ccccc1[C@@H](CCN([C@H](C)c1ccccc1)[C@@H](C)c1ccccc1)c1ccc(OC(C)C)cc1 ; COc1ccccc1[C@@H](CCN([C@H](C)c1ccccc1)[C@@H](C)c1ccccc1)c1ccc(OC(C)C)cc1]

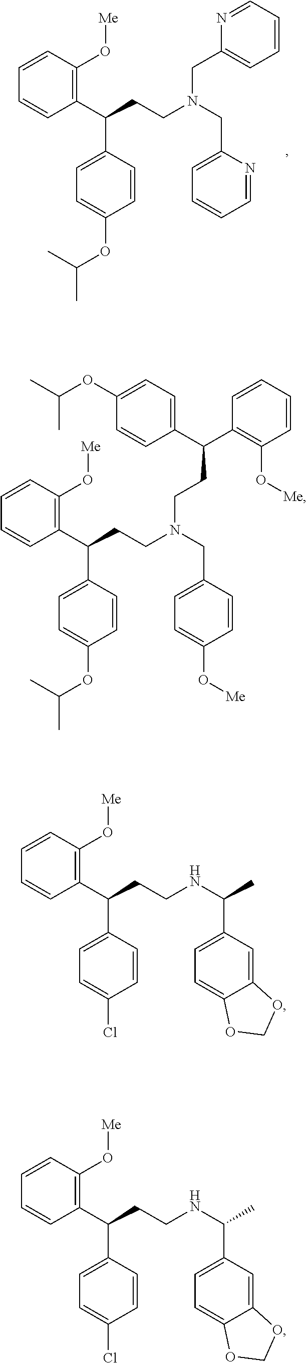 [SMILES: COc1ccccc1[C@@H](CCN(Cc1ccccn1)Cc1ccccn1)c1ccc(OC(C)C)cc1 ; COc1ccc(CN(CC[C@@H](c2ccc(OC(C)C)cc2)c2ccccc2OC)CC[C@@H](c2ccc(OC(C)C)cc2)c2ccccc2OC)cc1 ; COc1ccccc1[C@@H](CCN[C@@H](C)c1ccc2c(c1)OCO2)c1ccc(Cl)cc1 ; COc1ccccc1[C@@H](CCN[C@H](C)c1ccc2c(c1)OCO2)c1ccc(Cl)cc1]

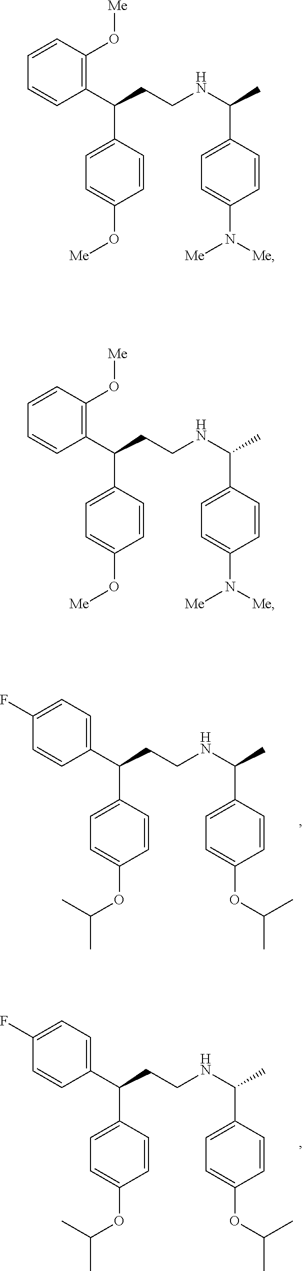 [SMILES: COc1ccc([C@H](CCN[C@@H](C)c2ccc(N(C)C)cc2)c2ccccc2OC)cc1 ; COc1ccc([C@H](CCN[C@H](C)c2ccc(N(C)C)cc2)c2ccccc2OC)cc1 ; CC(C)Oc1ccc([C@H](CCN[C@@H](C)c2ccc(OC(C)C)cc2)c2ccc(F)cc2)cc1 ; CC(C)Oc1ccc([C@H](CCN[C@H](C)c2ccc(OC(C)C)cc2)c2ccc(F)cc2)cc1]

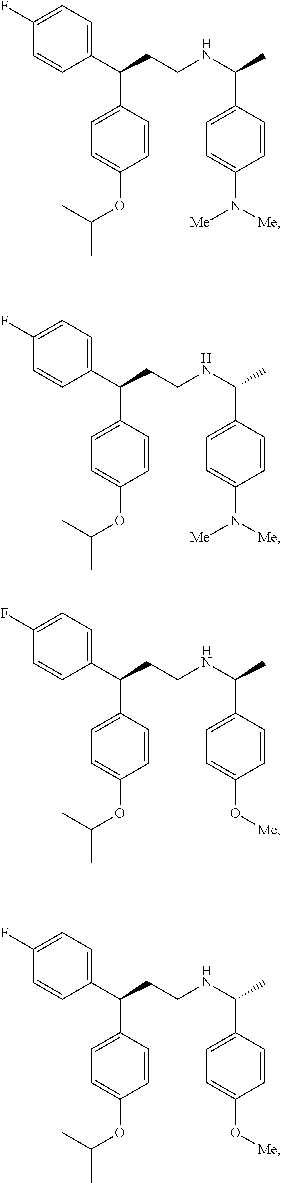 [SMILES: CC(C)Oc1ccc([C@H](CCN[C@@H](C)c2ccc(N(C)C)cc2)c2ccc(F)cc2)cc1 ; CC(C)Oc1ccc([C@H](CCN[C@H](C)c2ccc(N(C)C)cc2)c2ccc(F)cc2)cc1 ; COc1ccc([C@H](C)NCC[C@H](c2ccc(F)cc2)c2ccc(OC(C)C)cc2)cc1 ; COc1ccc([C@@H](C)NCC[C@H](c2ccc(F)cc2)c2ccc(OC(C)C)cc2)cc1]

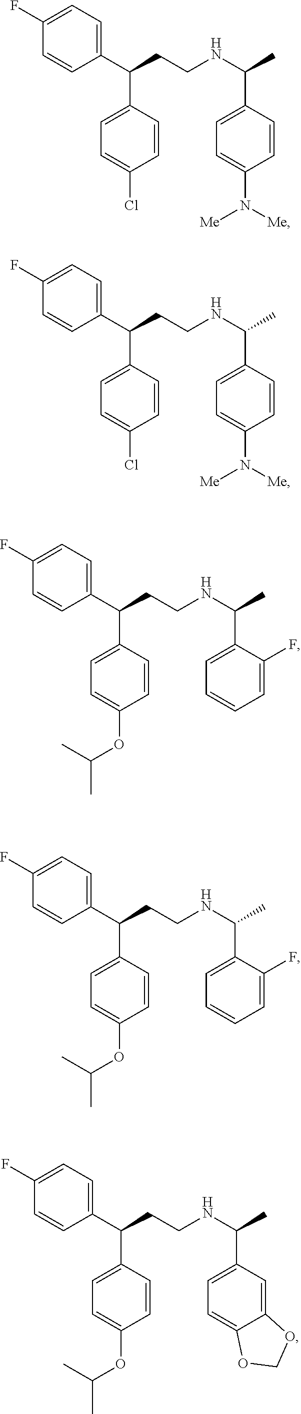 [SMILES: C[C@H](NCC[C@H](c1ccc(F)cc1)c1ccc(Cl)cc1)c1ccc(N(C)C)cc1 ; C[C@@H](NCC[C@H](c1ccc(F)cc1)c1ccc(Cl)cc1)c1ccc(N(C)C)cc1 ; CC(C)Oc1ccc([C@H](CCN[C@@H](C)c2ccccc2F)c2ccc(F)cc2)cc1 ; CC(C)Oc1ccc([C@H](CCN[C@H](C)c2ccccc2F)c2ccc(F)cc2)cc1 ; CC(C)Oc1ccc([C@H](CCN[C@@H](C)c2ccc3c(c2)OCO3)c2ccc(F)cc2)cc1]

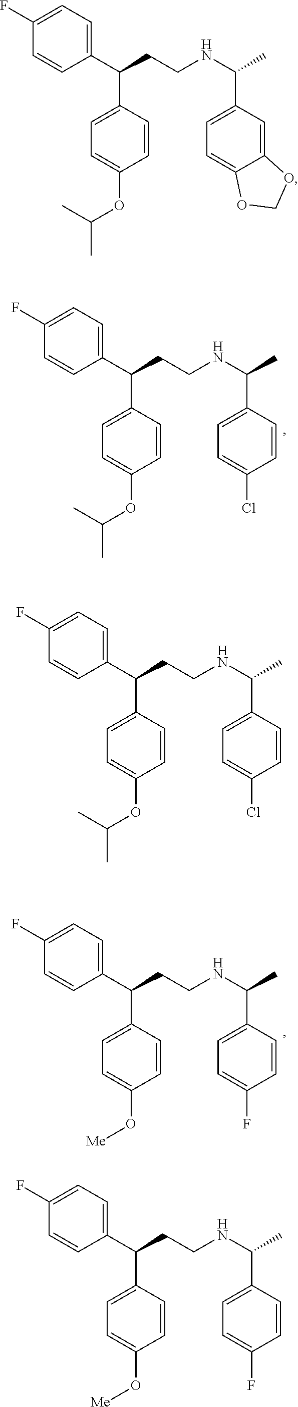 [SMILES: CC(C)Oc1ccc([C@H](CCN[C@H](C)c2ccc3c(c2)OCO3)c2ccc(F)cc2)cc1 ; CC(C)Oc1ccc([C@H](CCN[C@@H](C)c2ccc(Cl)cc2)c2ccc(F)cc2)cc1 ; CC(C)Oc1ccc([C@H](CCN[C@H](C)c2ccc(Cl)cc2)c2ccc(F)cc2)cc1 ; COc1ccc([C@H](CCN[C@@H](C)c2ccc(F)cc2)c2ccc(F)cc2)cc1 ; COc1ccc([C@H](CCN[C@H](C)c2ccc(F)cc2)c2ccc(F)cc2)cc1]

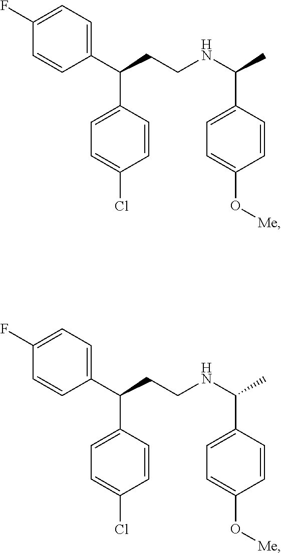 [SMILES: COc1ccc([C@H](C)NCC[C@H](c2ccc(F)cc2)c2ccc(Cl)cc2)cc1 ; COc1ccc([C@@H](C)NCC[C@H](c2ccc(F)cc2)c2ccc(Cl)cc2)cc1]

or a pharmaceutically acceptable salt and/or solvate thereof.
AB. The compound of any one of Paragraphs A-Y, wherein the compound is

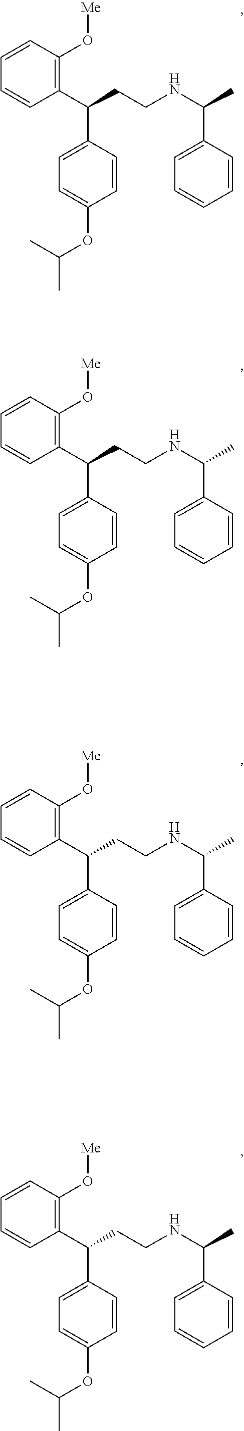 [SMILES: COc1ccccc1[C@@H](CCN[C@@H](C)c1ccccc1)c1ccc(OC(C)C)cc1 ; COc1ccccc1[C@@H](CCN[C@H](C)c1ccccc1)c1ccc(OC(C)C)cc1 ; COc1ccccc1[C@H](CCN[C@H](C)c1ccccc1)c1ccc(OC(C)C)cc1 ; COc1ccccc1[C@H](CCN[C@@H](C)c1ccccc1)c1ccc(OC(C)C)cc1]

pharmaceutically acceptable salt and/or solvate thereof.
AC. A composition comprising
   a compound of any one of Paragraphs A-AB; and
   a pharmaceutically acceptable carrier.
AD. A pharmaceutical composition comprising
   an effective amount of a compound of any one of Paragraphs A-AB for treating pathogenic cellular proliferation, angiogenesis, cancer, metastatic disease, and/or a pathogenic vascular proliferative disease in a subject; and
   a pharmaceutically acceptable carrier.
AE. The pharmaceutical composition of Paragraph AD, wherein the pathogenic vascular proliferative disease comprises pathogenic neovascularization associated with a cancer disease or condition.
AF. The pharmaceutical composition of Paragraph AD or Paragraph AE, wherein the pathogenic vascular proliferative disease comprises an ocular disease.
AG. The pharmaceutical composition of Paragraph AF, wherein the ocular disease is selected from the group consisting of age-related macular degeneration (AMD), diabetic retinopathy, retinopathy of prematurity, sickle cell retinopathy, retinal venous occlusive disease, central retinal vein occlusion (CRVO), branch retinal vein occlusion (BRVO), neovascular macular degeneration or an ocular cancer.
AH. The pharmaceutical composition of any one of Paragraphs AD-AG, wherein the pathogenic vascular proliferative disease comprises age-related macular degeneration (AMD).
AI. The pharmaceutical composition of any one of Paragraphs AD-AH, wherein the pathogenic vascular proliferative disease comprises wet, exudative age-related macular degeneration.
AJ. The pharmaceutical composition of any one of Paragraphs AD-AI, wherein the cancer comprises cholangiocarcinoma, triple negative breast cancer, or colorectal cancer.
AK. The pharmaceutical composition of any one of Paragraphs AD-AJ, wherein the pharmaceutical composition is formulated for parenteral administration, intravenous administration, subcutaneous administration, and/or oral administration.
AL. The pharmaceutical composition of any one of Paragraphs AD-AK, wherein the pharmaceutically acceptable carrier comprises 2-hydroxypropyl-β-cyclodextrin.
AM. The pharmaceutical composition of any one of Paragraphs AD-AL, wherein the pharmaceutical composition is formulated for mammalian subject suffering a neoplasm.
AN. The pharmaceutical composition of any one of Paragraphs AD-AM, wherein the pharmaceutical composition is formulated for a mammalian subject suffering or presenting with a history of neoplasm.
AO. The pharmaceutical composition of any one of Paragraphs AD-AN, wherein the pharmaceutical composition is formulated for a mammalian subject suffering or previously treated into clinical remission for a neoplasm.
AP. The pharmaceutical composition of any one of Paragraphs AD-AO, wherein the pharmaceutical composition is formulated for a mammalian subject suffering a condition mediated or contributed to by pathogenic neovascularization.
AQ. A method for treating a condition in a subject, the method comprising administering a compound of any one of Paragraphs A-AB to the subject in an amount effective to treat the condition, wherein the condition comprises one or more of pathogenic cellular proliferation, angiogenesis, cancer, metastatic disease, and/or a pathogenic vascular proliferative disease.
AR. The method of Paragraph AQ, wherein the pathogenic vascular proliferative disease comprises pathogenic neovascularization associated with a cancer disease or condition.
AS. The method of Paragraph AQ or Paragraph AR, wherein the pathogenic vascular proliferative disease comprises an ocular disease.
AT. The method of any one of Paragraphs AQ-AS, wherein the pathogenic vascular proliferative disease is selected from the group consisting of age-related macular degeneration (AMD), diabetic retinopathy, retinopathy of prematurity, sickle cell retinopathy, retinal venous occlusive disease, central retinal vein occlusion (CRVO), branch retinal vein occlusion (BRVO), neovascular macular degeneration or an ocular cancer.
AU. The method of any one of Paragraphs AQ-AT, wherein the pathogenic vascular proliferative disease comprises age-related macular degeneration (AMD).
AV. The method of any one of Paragraphs AQ-AU, wherein the pathogenic vascular proliferative disease comprises wet, exudative age-related macular degeneration.
AW. The method of any one of Paragraphs AQ-AV, wherein the cancer comprises cholangiocarcinoma, triple negative breast cancer, or colorectal cancer.
AX. The method of any one of Paragraphs AQ-AW, wherein the method comprises administering the compound by parenteral administration, intravenous administration, subcutaneous administration, and/or oral administration.

Other embodiments are set forth in the following claims, along with the full scope of equivalents to which such claims are entitled.

1. A compound according to Formula I 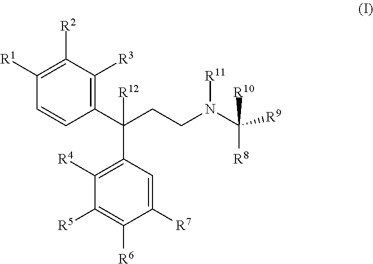
or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹³)(R¹⁴);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R⁵)(R¹);
R⁸ is aryl or heteroaryl;
R⁹ and R¹⁰ are each independently H, C₁-C₃ alkyl, or trifluoromethyl;
R¹¹ is H, C₁-C₃ alkyl, aralkyl, or heteroaralkyl;
at least one of R⁹, R¹⁰, and R¹¹ is not H;
R¹² is H, C₁-C₃ alkyl, or fluoro; and
R¹³, R¹⁴, R¹⁵, and R¹⁶ are each independently C₁-C₃ alkyl.

2. (canceled)

3. The compound of claim 1, wherein R¹, R², and R³ are each independently H, C₁-C₃ alkyl, C₁-C₃ alkoxy, halo, or —N(Me)₂.

4. The compound of claim 1, wherein R¹, R², and R³ are each independently H, methyl, methoxy, isopropyl, isopropoxy, fluoro, or —N(Me)₂.

5. The compound of claim 1, wherein R³ is methoxy.

6. (canceled)

7. The compound of claim 1, wherein R⁴, R⁵, R⁶, and R⁷ are each independently H, C₁-C₃ alkyl, C₁-C₃ alkoxy, halo, or —N(Me)₂.

8. The compound of claim 1, wherein R⁴, R⁵, R⁶, and R⁷ are each independently H, methyl, methoxy, isopropyl, isopropoxy, fluoro, or —N(Me)₂.

9. The compound of claim 1, wherein R⁶ is isopropoxy.

10. The compound of claim 1, where the compound is of Formula IA 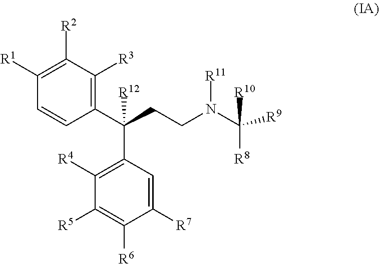
or a pharmaceutically acceptable salt and/or solvate thereof.

11. The compound of claim 1, wherein the compound is of Formula IB 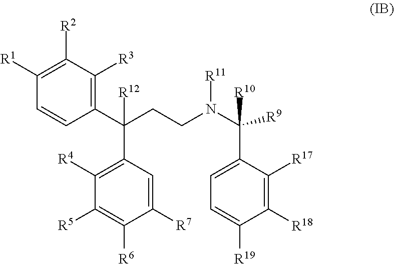
or a pharmaceutically acceptable salt and/or solvate thereof, wherein
R¹⁷, R¹⁸, and R¹⁹ are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido.

12.-14. (canceled)

15. The compound of claim 11, wherein at least one of R¹⁷, R¹⁸, and R¹⁹ is not H.

16. The compound of claim 11, wherein at least one of R¹⁷, R¹⁸, and R¹⁹ is C₁-C₃ alkoxy, trifluoromethyl, trifluoromethoxy, pentafluorosulfanyl, halo, aryloxy, aryloyl, hydroxyl, amino, or amido.

17. The compound of claim 11, wherein R¹⁷, R¹⁸, and R¹⁹ are each independently H.

18. The compound of claim 1, wherein R¹¹ is 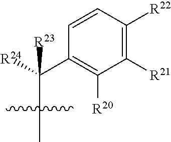
wherein
R²⁰, R²¹, and R²² are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido; and
R²³ and R²⁴ are each independently H, C₁-C₃ alkyl, trifluoromethyl, or aryl, provided at least one of R²³ and R²⁴ is not aryl.

19.-24. (canceled)

25. The compound of claim 1, wherein the compound is 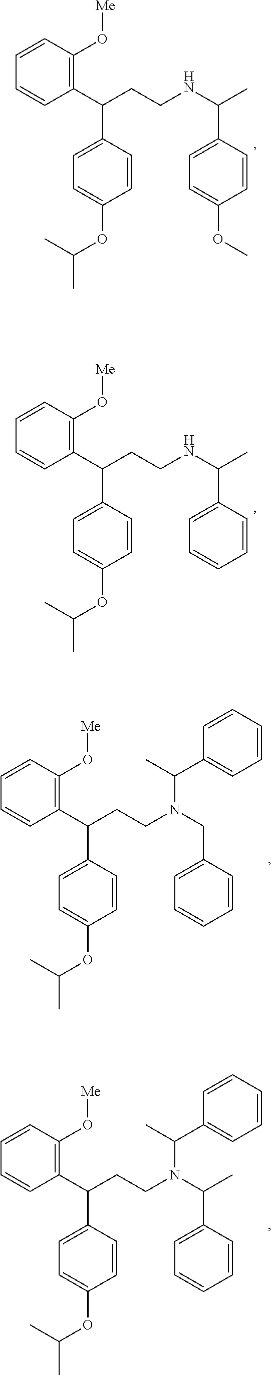 [SMILES: COc1ccc(C(C)NCCC(c2ccc(OC(C)C)cc2)c2ccccc2OC)cc1 ; COc1ccccc1C(CCNC(C)c1ccccc1)c1ccc(OC(C)C)cc1 ; COc1ccccc1C(CCN(Cc1ccccc1)C(C)c1ccccc1)c1ccc(OC(C)C)cc1 ; COc1ccccc1C(CCN(C(C)c1ccccc1)C(C)c1ccccc1)c1ccc(OC(C)C)cc1] 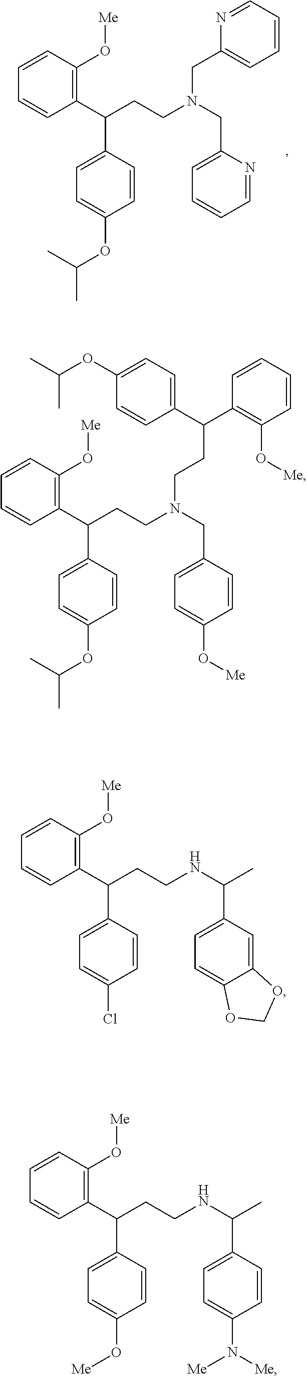 [SMILES: COc1ccccc1C(CCN(Cc1ccccn1)Cc1ccccn1)c1ccc(OC(C)C)cc1 ; COc1ccc(CN(CCC(c2ccc(OC(C)C)cc2)c2ccccc2OC)CCC(c2ccc(OC(C)C)cc2)c2ccccc2OC)cc1 ; COc1ccccc1C(CCNC(C)c1ccc2c(c1)OCO2)c1ccc(Cl)cc1 ; COc1ccc(C(CCNC(C)c2ccc(N(C)C)cc2)c2ccccc2OC)cc1] 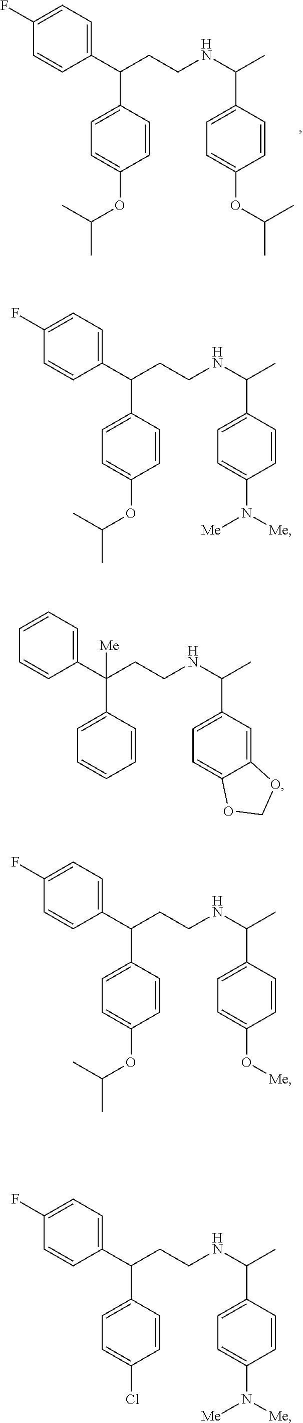 [SMILES: CC(C)Oc1ccc(C(C)NCCC(c2ccc(F)cc2)c2ccc(OC(C)C)cc2)cc1 ; CC(C)Oc1ccc(C(CCNC(C)c2ccc(N(C)C)cc2)c2ccc(F)cc2)cc1 ; CC(NCCC(C)(c1ccccc1)c1ccccc1)c1ccc2c(c1)OCO2 ; COc1ccc(C(C)NCCC(c2ccc(F)cc2)c2ccc(OC(C)C)cc2)cc1 ; CC(NCCC(c1ccc(F)cc1)c1ccc(Cl)cc1)c1ccc(N(C)C)cc1] 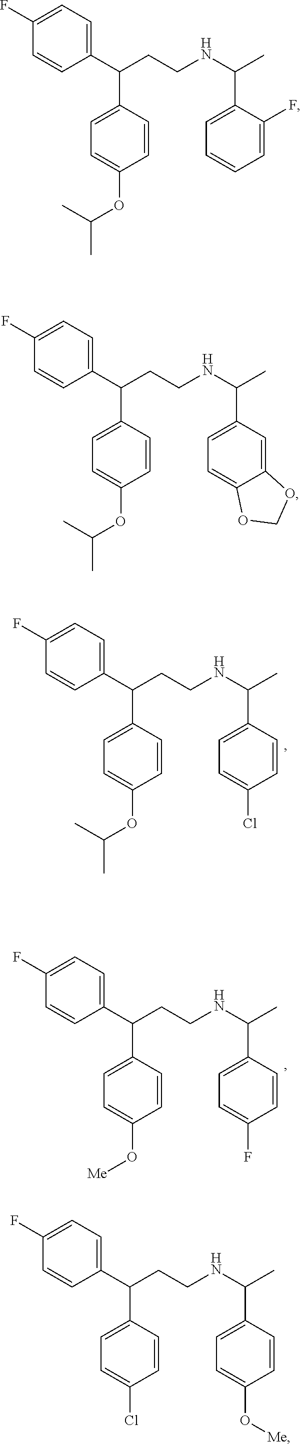 [SMILES: CC(C)Oc1ccc(C(CCNC(C)c2ccccc2F)c2ccc(F)cc2)cc1 ; CC(C)Oc1ccc(C(CCNC(C)c2ccc3c(c2)OCO3)c2ccc(F)cc2)cc1 ; CC(C)Oc1ccc(C(CCNC(C)c2ccc(Cl)cc2)c2ccc(F)cc2)cc1 ; COc1ccc(C(CCNC(C)c2ccc(F)cc2)c2ccc(F)cc2)cc1 ; COc1ccc(C(C)NCCC(c2ccc(F)cc2)c2ccc(Cl)cc2)cc1]
or a pharmaceutically acceptable salt and/or solvate thereof.

26. The compound of claim 1, wherein the compound is of Formula IC 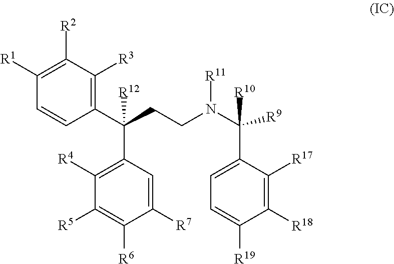
or a pharmaceutically acceptable salt and/or solvate thereof.

27. The compound of claim 1, wherein the compound is 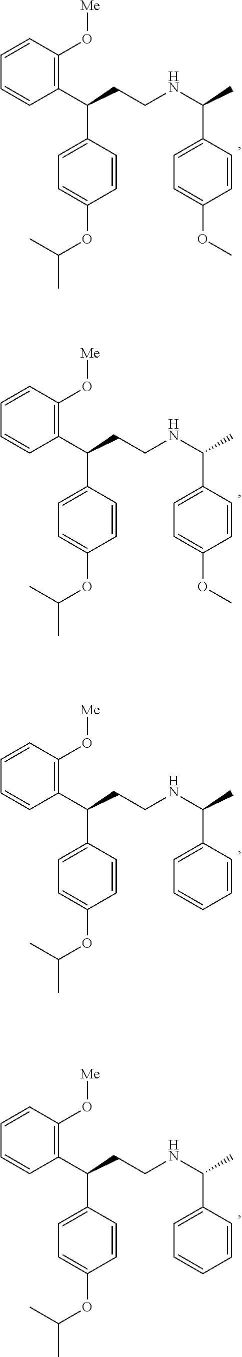 [SMILES: COc1ccc([C@H](C)NCC[C@@H](c2ccc(OC(C)C)cc2)c2ccccc2OC)cc1 ; COc1ccc([C@@H](C)NCC[C@@H](c2ccc(OC(C)C)cc2)c2ccccc2OC)cc1 ; COc1ccccc1[C@@H](CCN[C@@H](C)c1ccccc1)c1ccc(OC(C)C)cc1 ; COc1ccccc1[C@@H](CCN[C@H](C)c1ccccc1)c1ccc(OC(C)C)cc1] 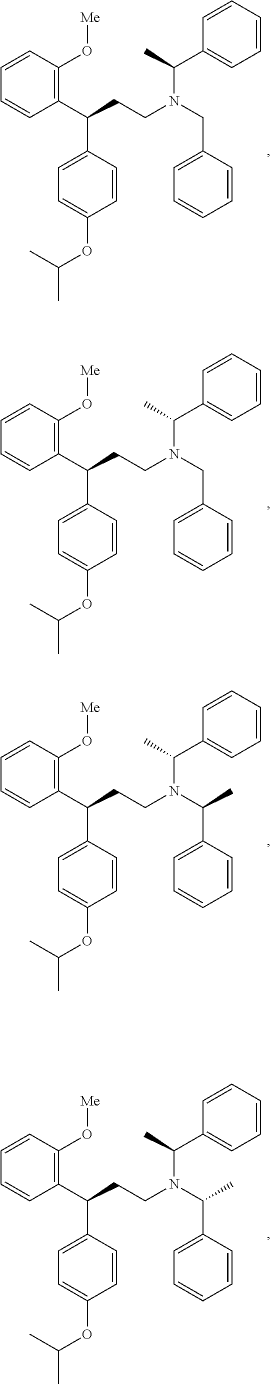 [SMILES: COc1ccccc1[C@@H](CCN(Cc1ccccc1)[C@@H](C)c1ccccc1)c1ccc(OC(C)C)cc1 ; COc1ccccc1[C@@H](CCN(Cc1ccccc1)[C@H](C)c1ccccc1)c1ccc(OC(C)C)cc1 ; COc1ccccc1[C@@H](CCN([C@H](C)c1ccccc1)[C@@H](C)c1ccccc1)c1ccc(OC(C)C)cc1 ; COc1ccccc1[C@@H](CCN([C@H](C)c1ccccc1)[C@@H](C)c1ccccc1)c1ccc(OC(C)C)cc1] 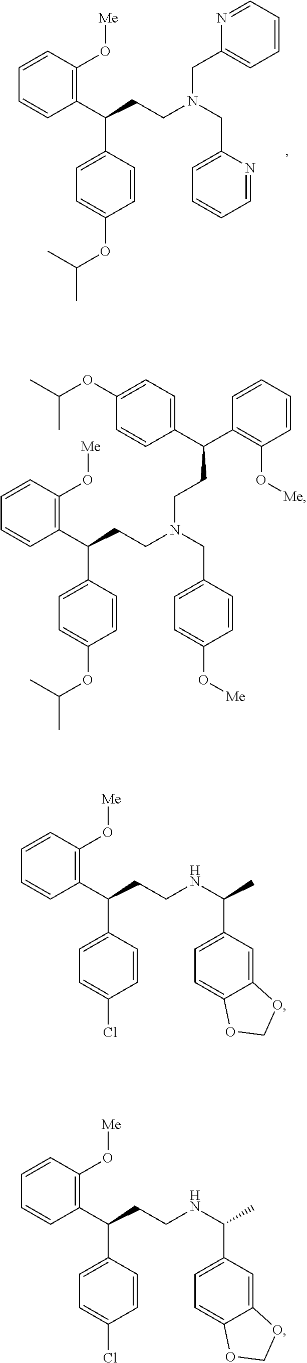 [SMILES: COc1ccccc1[C@@H](CCN(Cc1ccccn1)Cc1ccccn1)c1ccc(OC(C)C)cc1 ; COc1ccc(CN(CC[C@@H](c2ccc(OC(C)C)cc2)c2ccccc2OC)CC[C@@H](c2ccc(OC(C)C)cc2)c2ccccc2OC)cc1 ; COc1ccccc1[C@@H](CCN[C@@H](C)c1ccc2c(c1)OCO2)c1ccc(Cl)cc1 ; COc1ccccc1[C@@H](CCN[C@H](C)c1ccc2c(c1)OCO2)c1ccc(Cl)cc1] 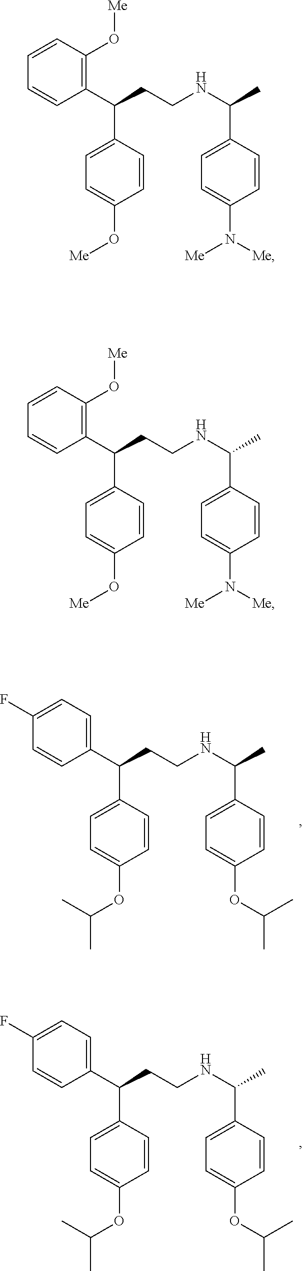 [SMILES: COc1ccc([C@H](CCN[C@@H](C)c2ccc(N(C)C)cc2)c2ccccc2OC)cc1 ; COc1ccc([C@H](CCN[C@H](C)c2ccc(N(C)C)cc2)c2ccccc2OC)cc1 ; CC(C)Oc1ccc([C@H](CCN[C@@H](C)c2ccc(OC(C)C)cc2)c2ccc(F)cc2)cc1 ; CC(C)Oc1ccc([C@H](CCN[C@H](C)c2ccc(OC(C)C)cc2)c2ccc(F)cc2)cc1] 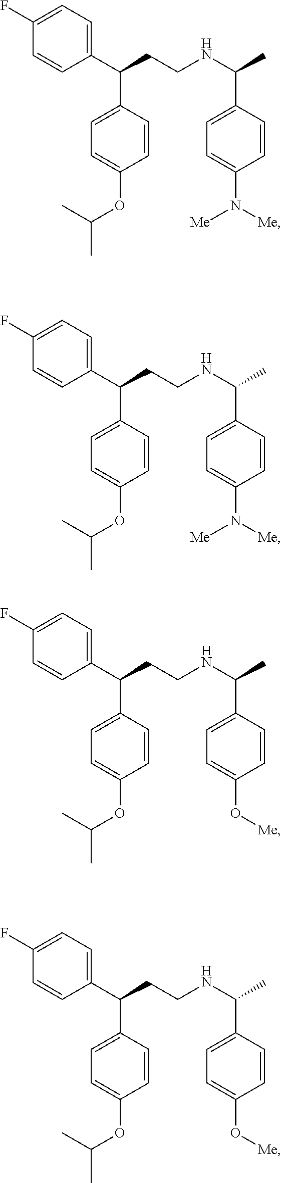 [SMILES: CC(C)Oc1ccc([C@H](CCN[C@@H](C)c2ccc(N(C)C)cc2)c2ccc(F)cc2)cc1 ; CC(C)Oc1ccc([C@H](CCN[C@H](C)c2ccc(N(C)C)cc2)c2ccc(F)cc2)cc1 ; COc1ccc([C@H](C)NCC[C@H](c2ccc(F)cc2)c2ccc(OC(C)C)cc2)cc1 ; COc1ccc([C@@H](C)NCC[C@H](c2ccc(F)cc2)c2ccc(OC(C)C)cc2)cc1] 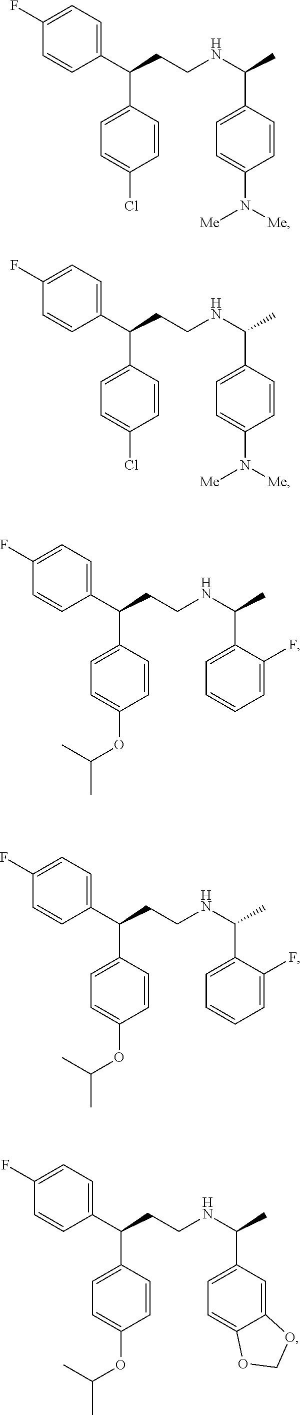 [SMILES: C[C@H](NCC[C@H](c1ccc(F)cc1)c1ccc(Cl)cc1)c1ccc(N(C)C)cc1 ; C[C@@H](NCC[C@H](c1ccc(F)cc1)c1ccc(Cl)cc1)c1ccc(N(C)C)cc1 ; CC(C)Oc1ccc([C@H](CCN[C@@H](C)c2ccccc2F)c2ccc(F)cc2)cc1 ; CC(C)Oc1ccc([C@H](CCN[C@H](C)c2ccccc2F)c2ccc(F)cc2)cc1 ; CC(C)Oc1ccc([C@H](CCN[C@@H](C)c2ccc3c(c2)OCO3)c2ccc(F)cc2)cc1] 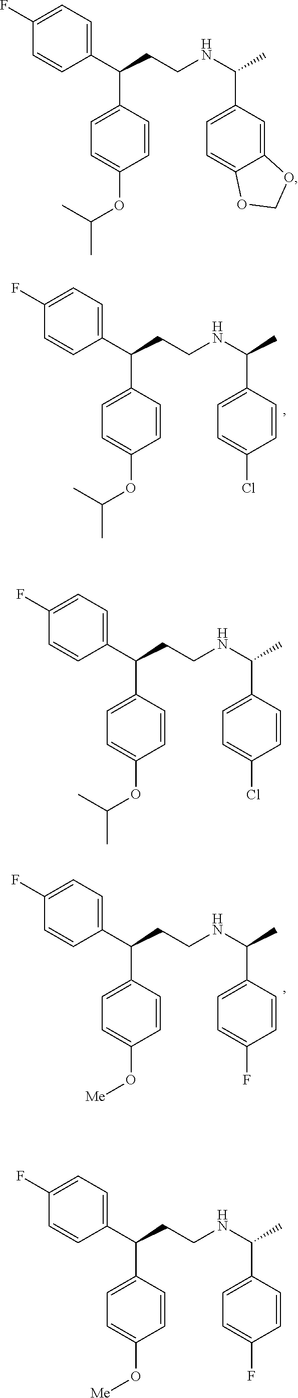 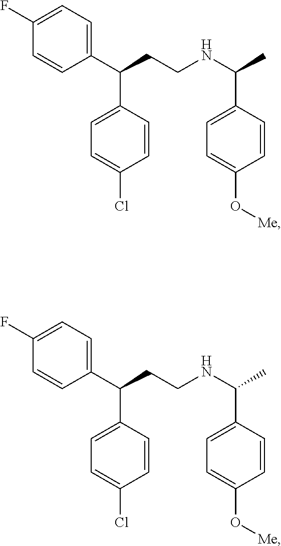 [SMILES: COc1ccc([C@H](C)NCC[C@H](c2ccc(F)cc2)c2ccc(Cl)cc2)cc1 ; COc1ccc([C@@H](C)NCC[C@H](c2ccc(F)cc2)c2ccc(Cl)cc2)cc1]
or a pharmaceutically acceptable salt and/or solvate thereof.

28. The compound of claim 1, wherein the compound is 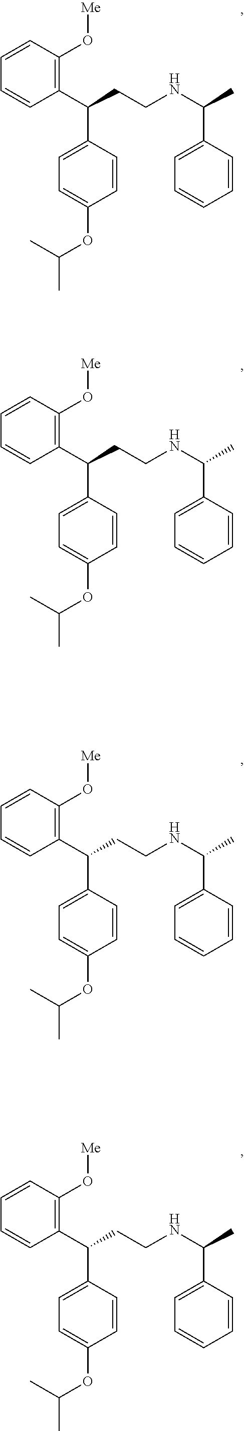 [SMILES: COc1ccccc1[C@@H](CCN[C@@H](C)c1ccccc1)c1ccc(OC(C)C)cc1 ; COc1ccccc1[C@@H](CCN[C@H](C)c1ccccc1)c1ccc(OC(C)C)cc1 ; COc1ccccc1[C@H](CCN[C@H](C)c1ccccc1)c1ccc(OC(C)C)cc1 ; COc1ccccc1[C@H](CCN[C@@H](C)c1ccccc1)c1ccc(OC(C)C)cc1]
or a pharmaceutically acceptable salt and/or solvate thereof.

29. A composition comprising
a compound of claim 1; and
a pharmaceutically acceptable carrier.

30. A pharmaceutical composition comprising
an effective amount of a compound of claim 1 for treating pathogenic cellular proliferation, angiogenesis, cancer, metastatic disease, and/or a pathogenic vascular proliferative disease in a subject; and
a pharmaceutically acceptable carrier.

31.-42. (canceled)

43. A method for treating a condition in a subject, the method comprising administering a compound of claim 1 to the subject in an amount effective to treat the condition, wherein the condition comprises one or more of pathogenic cellular proliferation, angiogenesis, cancer, metastatic disease, and/or a pathogenic vascular proliferative disease.

44.-50. (canceled)
